(19) United States
(12) Patent Application Publication
(10) Pub. No.: US 2003/0113324 A1
(43) Pub. Date: Jun. 19, 2003

(54) Neuropilin/VEGF-C/VEGFR-3 materials and methods

(76) Inventors: Kari Alitalo, Helsinki (FI); Marika Karkkainen, Helsinki (FI); Kaisa Karila, Espoo (FI)

Correspondence Address: MARSHALL, GERSTEIN & BORUN, 6300 SEARS TOWER, 233 SOUTH WACKER, CHICAGO, IL, 60606-6357, US

(21) Appl. No.: 10/262,538

(22) Filed: Sep. 30, 2002

Related U.S. Application Data
(60) Provisional application No. 60/326,326, filed on Oct. 1, 2001.

(51) Int. Cl. G01N 33/53; A61K 39/395

(52) U.S. Cl. 424/145.1; 435/7.1

(57) ABSTRACT

The present invention relates to identifying modulators of VEGF-C or VEGF-D ligand binding to the nervous system transmembrane protein neuropilin-2 and materials and methods for detecting said modulators.

This application claims priority benefit of U.S. Provisional Application No. 60/326,326, filed Oct. 1, 2001.

### FIELD OF THE INVENTION

The present invention provides materials and methods relating to cellular and molecular biology and medicine, particularly in the areas of vascularization and angiogenesis and the interactions of the vascular system with the nervous system.

### BACKGROUND OF THE INVENTION

Interactions of the neuropilin receptor proteins with their ligands in the collapsin/semaphorin family of molecules promotes development of neuronal growth cones and axon guidance, the process which regulates the paths of extending axons during the development of neuronal tissue. Improper retraction of the neural growth cones leads to excess, unwanted innervation of tissue.

Collapsin/semaphorin proteins belong to a family of molecules containing a characteristic semaphorin domain of approximately 500 amino acids in the amino terminus. Over 20 members of the semaphorin family are currently known, both secreted and membrane bound forms, which can be divided into six different subgroups based on primary protein structure. Both secreted and membrane bound semaphorins bind to their receptors as disulfide linked homodimers, and the cytoplasmic tail of membrane bound semaphorins can induce clustering of these ligands in the cell membrane.

Class III semaphorins, secreted proteins which contain the semaphorin domain followed by a C2-type immunoglobulin like domain, have been found to be integrally involved in the repulsion and collapse of neuronal growth cones, a process which prevents improper innervation of dorsal root ganglia, sympathetic neurons, and both cranial and spinal neurons.

Recently, two receptors for the class III semaphorins were identified, neuropilin-1(NRP-1) (Kolodkin et al, Cell. 90:753-762. 1997 and He et al, Cell. 90:739-51. 1997) and neuropilin-2 (NRP-2) (Chen et al, Neuron, 19:547. 1997). Neuropilin-1, a type-I membrane protein originally isolated from the Xenopus nervous system, was identified by semaphorin III receptor expression cloning, as a high affinity receptor for Sema III and other semaphorin family members. Further analysis by PCR using sequences homologous to neuropilin-1 identified a related receptor, neuropilin-2, which shows approximately 44% homology to NRP-1 throughout the entire protein length.

The extracellular portion of both NRP-1 and NRP-2 shows an interesting mix of cell binding domains, possessing five distinct protein domains designated a1/a2, b1/b2, and c. The a1/a2 (CUB) domains resemble protein sequences found in complement components C1r and Cs while the b1/b2 domains are similar to domains found in coagulation factors V and VIII. The central portion of the c domain, similar to the meprin/A5/mu-phosphotase (MAM) homology domain, is important for neuropilin dimerization. The intracellular region of neuropilins contains a transmembrane domain and a short, highly conserved cytoplasmic tail of ˜43 amino acids that possesses no known catalytic activity to date. Both the a1/a2 and b1/b2 domains are necessary to facilitate semaphorin binding to neuropilins.

Since the short cytoplasmic tail of neuropilins does not possess signaling capabilities, neuropilins probably couple with other receptors to transmit intracellular signals as a result of semaphorin binding. Investigation of this scenario concluded that neuropilins interact with another family of semaphorin receptors, the plexins, which possess a cytoplasmic tail containing a sex-plexin domain capable of undergoing phosphorylation and initiating downstream signaling cascades (Tamagnone et al Trends in Cell Biol, 10:377-83. 2000). Plexins were originally isolated as orphan receptors for membrane bound semaphorins, and plexins alone are incapable of binding secreted semaphorins such as those in the class III subfamily. A great deal of evidence now demonstrates that class III semaphorin binding is mediated through a receptor complex which includes homo- or heterodimeric neuropilins and a plexin molecule needed to transduce intracellular signals. Interactions of plexins with neuropilins confers specificity of semaphorin binding and can also increase the binding affinity of these ligands. Signaling of semaphorins through their receptors is reviewed in Fujisawa et al, (Current Opinion in Neurobiology, 8:587. 1998) and Tamagnone et al, (Trends in Cell Biol, 10:377. 2000).

Neuropilin-1 (Tagaki et al., Neuron 7:295-307. 1991; Fujisawa et al., Cell Tissue Res. 290:465-70. 1997), a 140 kD protein whose gene is localized to chromosome 10p12 (Rossingnol et al., Genomics 57:459-60. 1999), is expressed in a wide variety of tissues during development, including nervous tissue, capillaries and vessels of the cardiovascular system, and skeletal tissue, and persists in many adult tissues, most notably the placenta and heart. In addition to binding Sema3A, NRP-1 also binds several other semaphorin family members including Sema3B, Sema3C (SemaE), and Sema3F (SemaIV) (with low affinity) (He et al., Cell 90:739-51. 1997; Kolodkin et al.,Cell 90:753-62. 1997). Mice homozygous mutant at the NRP-1 locus demonstrate defects not only in axonal guidance but also show altered vascularization in the brain and defects in the formation of large vessels of the heart (Kawasaki et al, Development 126:4895. 1990). Interestingly, NRP-1 overexpression in embryos leads to excess capillary and vessel formation and hemorrhaging, implicating a role for NRP-1 in vascular development (Kitsukawa et al, Development, 121:4309. 1995).

Recent evidence shows that neuropilin-1 can act as a receptor for an isoform of vascular endothelial growth factor (VEGF/VEGF-A) (Soker et al, Cell 92:735. 1998), which is a key mediator of vasculogenesis and angiogenesis in embryonic development (reviewed in Robinson et al, J. Cell Science. 114:853-65) and also plays a significant role in tumor angiogenesis. Binding of VEGF to receptor tyrosine kinases (RTK) VEGFR-1 and VEGFR-2 facilitates vascular development. Both the non-heparin dependent VEGF₁₂₁ isoform and the heparin-binding VEGF₁₆₅ bind VEGFR-2 with the same affinity in vitro, but do not elicit equivalent biochemical responses, indicating that additional factors mediate VEGFR-2 activation (Whitaker et al, J Bio Chem. 276:25520-31. 2001). Analysis of the binding of several splice variants of VEGF reveal that NRP-1 does not bind the VEGF₁₂₁ isoform but selectively binds the VEGF₁₆₅ variant in a heparin-dependent manner within the b domain of NRP-1 (Giger et al, Neuron 21:1079-92. 1998). NRP-1 demonstrates a binding affinity for the VEGF₁₆₅ isoform comparable to that of it's Sema3A ligand. This differential affinity of NRP-1 for VEGF₁₆₅ may explain the signaling capabilities of this splice variant over the non-heparin binding VEGF₁₂₁ and may indicate that neuropilin-1 interacts with VEGFR-2 as a co-receptor in VEGF binding (Whitaker et al., 2001), similar to its role in plexin/semaphorin complexes. VEGF₁₆₅ binds NRP-1 through VEGF exon 7, which confers heparin binding affinity to this molecule, and is lacking in the VEGF₁₂₁ isoform. NRP-1 also binds other VEGF family members, VEGF-B and placenta growth factor (PlGF-2) (Migdal et al, J. Biol.Chem. 273:22272-78. 1998; Makinen et al, J. Biol. Chem. 274: 21217-222. 1999).

Neuropilin-2 (Chen et al, Neuron 19:547-59. 1997), a 120 kD protein whose gene is localized to chromosome 2q34 (Rossingnol et al., Genomics 57:459-60. 1999), exhibits similar tissue distribution in the developing embryo as neuropilin-1, but does not appear to be expressed in endothelial cells of capillaries (Chen et al, Neuron 19:547-59. 1997). NRP-2 is also a semaphorin receptor, binding Sema3F with high affinity, Sema3C with affinity comparable to Sema3C/NRP-1 binding, NRP-2 also appears to interact with very low affinity to Sema3A (Kolodkin et al.,Cell 90:753-62. 1997). NRP-2 deficient mice survive embryogenesis with no apparent vascular defects, but exhibit defects in the Sema3F-dependent formation of sympathetic and hippocampal neurons and defects in axonal projections in the peripheral and central nervous systems, implicating NRP-2 in axonal guidance (Chen et al, Neuron 25:43-56. 2000; Giger et al, Neuron 25:29-41. 2000) and suggesting distinct roles for NRP-1 and NRP-2 in development. NRP-2 expression has also been noted in sites that innervate smooth muscle cells such as mesentery, muscular, and submucosal plexuses (Cohen et al, Biochem Biophy Res Comm. 284:395-403. 2001).

Experimental evidence establishes that, similar to NRP-1, neuropilin-2 preferentially binds VEGF₁₆₅, and shows additional binding to the VEGF₁₄₅ isoform, another heparin-binding splice variant of VEGF (Gluzman-Poltorak et al, J. Biol Chem. 275:18040-45. 2000). Neuropilin-2 interaction with the VEGF₁₄₅ splice variant, which lacks exon 7, is mediated through VEGF₁₄₅ exon 6 which, like exon 7, is capable of mediating heparin binding activity. VEGF₁₄₅ cannot bind NRP-1, which further supports the theory of differential functions for neuropilin-1 and neuropilin-2 in vascular development. VEGF₁₄₅ was originally isolated from carcinomas of the female reproductive tract (Pavelock et al, Endocrinology. 142: 613-22. 2001) where neuropilin-2 expression shows differential regulation in response to hormonal changes as compared to NRP-1 and VEGFR-2. The co-expression of both neuropilins, VEGFs, and VEGFRs in a particular cell type may be indicative of a potential receptor/ligand complex formation and needs to be investigated in greater detail.

VEGF/VEGFR interactions play an integral role in embryonic vasculogenesis and angiogenesis, as well as a role in adult tissue neovascularization during wound healing, remodeling of the female reproductive system, and tumor growth. Elucidating additional factors involved in the regulation of neovascularization and angiogenesis, as well as their roles in such processes, would aid in the development of therapies directed toward prevention of vascularization of solid tumors and induction of tumor regression, and induction of vascularization to promote faster, more efficient wound healing after injury, surgery, or tissue transplantation, or to treat ischemia by inducing angiogenesis and arteriogenesis of vessels that nourish the ischemic tissue. In fact, modulation of angiogenic processes may be instrumental in treatment or cure of many of the most significant diseases that plague humans in the developed world, such as cerebral infarction/bleeding, acute myocardial infarction and ischemia, and cancers. Modulation of neuronal growth also is instrumental in treatment of numerous congenital, degenerative, and trauma-related neurological conditions. The newfound interaction between neuropilins and VEGF provided one target for intervention at a molecular level for both neuronal and vascular diseases and conditions. However, the ability to develop targeted therapies is complicated by the existence of multiple binding partners for neuropilins. There exists a need to delineate molecules that bind neuropilins in order to permit identification of modulation of neuropilin activities and to optimize the specificity of such molecules to optimize therapies in areas of unwanted angiogenesis, as in cancers or solid tumor growth, and potentiate pro-angiogenic properties to promote and speed needed blood vessel growth, as in wound healing; and optimize therapies directed to neuronal growth and organization.

### SUMMARY OF THE INVENTION

The present invention addresses one or more needs in the art relating to modulation of angiogenic and nervous system growth and function, by identifying novel molecular interactions between neuropilins and VEGF-C molecules, and between neuropilins and VEGFR-3 molecules. These newly delineated interactions facilitate identification of novel materials and methods for modulating both angiogenic processes (including lymphangiogenic processes) and processes involved in neural cell regeneration. The newly delineated interactions also facilitate better therapeutic targeting by permitting design of molecules that modulate single receptor-ligand interactions highly selectively, or molecules that modulate multiple interactions.

For example, the discovery of VEGF-C-neuropilin interactions provides novel screening assays to identify new therapeutic molecules to modulate (up-regulate/activate/stimulate or downregulate/inhibit) VEGF-C-neuropilin interactions. Such molecules are useful as therapeutics (and/or as lead compounds) for diseases and conditions in which VEGF-C/neuropilin interactions have an influence, including those in which lymphatic or blood vessel growth play a role.

In one embodiment, the invention provides a method for identifying a modulator of binding between a neuropilin receptor and VEGF-C polypeptide comprising steps of:

a) contacting a neuropilin composition that comprises a neuropilin polypeptide with a VEGF-C composition that comprises a VEGF-C polypeptide, in the presence and in the absence of a putative modulator compound;

b) detecting binding between neuropilin polypeptide and VEGF-C polypeptide in the presence and absence of the putative modulator; and

c) identifying a modulator compound based on a decrease or increase in binding between the neuropilin polypeptide and the VEGF-C polypeptide in the presence of the putative modulator compound, as compared to binding in the absence of the putative modulator compound.

In one variation, the method further includes a step (d) of making a modulator composition by formulating a modulator identified according to step (c) in a carrier, preferably a pharmaceutically acceptable carrier. A modulator so formulated is useful in animal studies and also as a therapeutic for administration to image tissues or treat diseases associated with neuropilin-VEGF-C interactions, wherein the administration of a compound could interfere with detrimental activity of these molecules, or promote beneficial activity. Thus, in still another variation, the method further includes a step (e) of administering the modulator composition to an animal that comprises cells that express the neuropilin receptor, and determining physiological effects of the modulator composition in the animal. The animal may be human, or any animal model for human medical research, or an animal of importance as livestock or pets. In a preferred variation, the animal (including humans) has a disease or condition characterized by aberrant neuropilin-2/VEGF-C biology, and the modulator improves the animal's state (e.g., by reducing disease symptoms, slowing disease progression, curing the disease, or otherwise improving clinical outcome).

Step (a) of the foregoing methods involves contacting a neuropilin composition with a VEGF-C composition in the presence and absence of a compound. By “neuropilin composition” is meant any composition that includes a whole neuropilin receptor polypeptide, or includes at least the portion of the neuropilin polypeptide needed for the particular assay—in this case the portion of the neuropilin polypeptide involved in VEGF-C binding. Exemplary neuropilin compositions include: (i) a composition comprising a purified polypeptide that comprises an entire neuropilin protein or that comprises a neuropilin receptor extracellular domain fragment that binds VEGF-C polypeptides; (ii) a composition containing phospholipid membranes that contain neuropilin receptor polypeptides on their surface; (iii) a living cell recombinantly modified to express increased amounts of a neuropilin receptor polypeptide on its surface (e.g., by inserting a neuropilin gene, preferably with an attached promoter, into a cell; or by amplifying an endogenous neuropilin gene; or by inserting an exogenous promoter or other regulatory sequence to up-regulate an endogenous neuropilin gene); and (iv) any isolated cell or tissue that naturally expresses the neuropilin receptor polypeptide on its surface. For certain assay formats, it may be desirable to bind the neuropilin molecule of interest (e.g., a composition comprising a polypeptide comprising a neuropilin receptor extracellular domain fragment) to a solid support such as a bead or assay plate well. “Neuropilin composition” is intended to include such structures as well. Likewise, fusion proteins are contemplated wherein the neuropilin polypeptide is fused to another protein (such as an antibody Fc fragment) to improve solubility, or to provide a marker epitope, or serve any other purpose. For other assay formats, soluble neuropilin peptides may be preferred. In one preferred variation, the neuropilin composition comprises a polypeptide comprising a neuropilin receptor extracellular domain fragment fused to an immunoglobulin Fc fragment. Although two family members are currently known, neuropilin-1 and neuropilin-2, practice of the invention with other neuropilin receptor family members that are subsequently discovered is contemplated. The neuropilin receptor chosen is preferably of vertebrate origin, more preferably mammalian, still more preferably primate, and still more preferably human. And, while it will be apparent that the assay will likely give its best results if the functional portion of the chosen neuropilin receptor is identical in amino acid sequence to the native receptor, it will be apparent that the invention can still be practiced if variations have been introduced in the neuropilin sequence that do not eliminate its VEGF-C binding properties. Use of variant sequences with at least 90%, 95%, 96%, 97%, 98%, or 99% amino acid identity is specifically contemplated.

VEGF-C molecules occur naturally as secreted factors that undergo several enzymatic cleavage reactions before release into the surrounding milieu. Thus, “VEGF-C composition” means any composition that includes a prepro-VEGF-C polypeptide, the intermediate and final cleavage products of prepro-VEGF-C, ΔNΔCVEGF-C, or includes at least the portion of the VEGF-C needed for the particular assay—in this case the portion involved in binding to a neuropilin receptor. Exemplary VEGF-C compositions include: (i) a composition comprising purified complete prepro-VEGF-C polypeptide or comprising a prepro-VEGF-C polypeptide fragment that binds the neuropilin receptor chosen for the assay; and (ii) conditioned media from a cell that secretes the VEGF-C protein. For certain assay formats, it may be desirable to bind the VEGF-C molecule of interest (e.g., a polypeptide comprising VEGF-C fragment) to a solid support such as a bead or assay plate well. “VEGF-C composition” is intended to include such structures as well. Likewise, fusion proteins are contemplated. The data provided herein establishes that isoforms of VEGF-C bind both neuropilin-1 and neuropilin-2. The VEGF-C polypeptide chosen is preferably of vertebrate origin, more preferably mammalian, still more preferably primate, and still more preferably human. In one embodiment the VEGF-C compositions comprises a fragment of human prepro-VEGF-C that contains amino acids 103-227 of SEQ. ID NO.: 24. In another embodiment, the VEGF-C composition comprises amino acids 32-227 of the human prepro-VEGF-C sequence of SEQ. ID NO.: 24. While it will be apparent that the assay will likely give its best results if the functional portion of the chosen VEGF-C is identical in amino acid sequence to the corresponding portion of the native VEGF-C, it will be apparent that the invention can still be practiced if variations have been introduced in the VEGF-C sequence that do not eliminate its neuropilin receptor binding properties. Use of variant sequences with at least 90%, 95%, 96%, 97%, 98%, or 99% amino acid identity is specifically contemplated.

The putative modulator compound that is employed in step (a) can be any organic or inorganic chemical or biological molecule or composition of matter that one would want to test for ability to modulate neuropilin-VEGF-C interactions. Since the most preferred modulators will be those that can be administered as therapeutics, it will be apparent that molecules with limited toxicity are preferred. However, toxicity can be screened in subsequent assays, and can be “designed out” of compounds by pharmaceutical chemists. Screening of chemical libraries such as those customarily kept by pharmaceutical companies, or combinatorial libraries, peptide libraries, and the like is specifically contemplated.

Step (b) of the above-described method includes detecting binding between neuropilin and VEGF-C in the presence and absence of the compound. Any technique for detecting intermolecular binding may be employed. Techniques that provide quantitative measurements of binding are preferred. For example, one or both of neuropilin/VEGF-C may comprise a label, such as a radioisotope, a fluorophore, a fluorescing protein (e.g., natural or synthetic green fluorescent proteins), a dye, an enzyme or substrate, or the like. Such labels facilitate quantitative detection with standard laboratory machinery and techniques. Immunoassays represent a common and highly effective body of techniques for detecting binding between two molecules.

When the neuropilin composition comprises a cell that expresses neuropilin naturally or recombinantly on its surface, it will often be possible to detect VEGF-C binding indirectly, e.g., by detecting or measuring a VEGF-C binding-induced physiological change in the cell. Such possible changes include phosphorylation of the neuropilin associated VEGF-receptor; cell chemotaxis; cell growth; DNA synthesis; changes in cellular morphology; ionic fluxes; or the like.

Step (c) of the outlined method involves identifying a modulator compound on the basis of increased or decreased binding between the neuropilin receptor polypeptide and the VEGF-C polypeptide in the presence of the putative modulator compound as compared to such binding in the absence of the putative modulator compound. Generally, more attractive modulators are those that will activate or inhibit neuropilin-VEGF-C binding at low concentrations, thereby permitting use of the modulators in a pharmaceutical composition at lower effective doses.

As described below in greater detail, the growth factor VEGF-D shares amino acid sequence similarity to VEGF-C, and is known to undergo similar proteolytic processing from a prepro-VEGF-D form into smaller, secreted growth factor forms, and is known to share two VEGFR receptors with VEGF-C, namely, VEGFR-3 and VEGFR-2. Due to these and other similarities, it is expected that VEGF-D binds neuropilins in a manner analogous to what has been shown with VEGF-C, and such binding may be confirmed with assays described in the examples (by substituting VEGF-D). Accordingly, as another aspect of the invention, practice of the above-described screening method (and other methods described in the ensuing paragraphs) is contemplated wherein VEGF-D polypeptides are employed in lieu of VEGF-C polypeptides. A detailed description of the human VEGF-D gene and protein are provided in Achen, et al., Proc. Nat'l Acad. Sci. U.S.A., 95(2): 548-553 (1998); International Patent Publication No. WO 98/07832, published Feb. 26, 1998; and in Genbank Accession No. AJ000185, all incorporated herein by reference.

In another embodiment, the invention provides a method for screening for selectivity of a modulator of VEGF-C biological activity. The term “selectivity”—when used herein to describe modulators—refers to the ability of a modulator to modulate one protein-protein interaction (e.g., VEGF-C binding with neuropilin-2) with minimal effects on the interaction of another protein-protein interaction of one or more of the binding pairs (e.g., VEGF-C binding with VEGFR-2, or VEGFR-3, or neuropilin-1). More selective modulators significantly alter the first protein-protein interaction with minimal effects on the other protein-protein interaction, whereas non-selective modulators will alter two or more protein-protein interactions. It will be appreciated that selectivity is of immense interest to the design of effective pharmaceuticals. For example, in some circumstances, it may be desirable to identify modulators that alter VEGF-C/neuropilin interactions but not semaphorin/neuropilin interactions, because one wishes to modulate vessel growth but not neurological growth. It may be desirable in some circumstances to non-selectively inhibit all VEGF-C related activities, e.g., in anti-tumor therapy. The molecular interactions identified herein permit novel screening assays to help identify the selectivity of modulators.

For example, VEGF-C molecules are also known ligands for the VEGFR-2 and VEGFR-3 tyrosine kinase receptors. VEGF-C/VEGFR-3 interactions appear to be integrally involved in the development and maintenance of lymphatic vasculature and may also be involved in cancer metastasis through the lymphatic system. In one instance it may be beneficial to modulate VEGF-C/neuropilin interactions specifically while in another instance it may be useful to selectively modulate the VEGF-C/VEGFR interactions. The present invention provides counterscreen assays that identify the selectivity of a modulator for neuropilin-VEGF-C binding or VEGF-C-VEGFR binding.

Thus, in one variation, the invention provides a method, comprising steps of:

a) contacting a VEGF-C composition with a neuropilin composition in the presence and in the absence of a compound and detecting binding between the VEGF-C and the neuropilin (in the compositions) in the presence and absence of the compound, wherein differential binding in the presence and absence of the compound identifies the compound as a modulator of binding between the VEGF-C and the neuropilin;

b) contacting a VEGF-C composition with a composition comprising a VEGF-C binding partner in the presence and in the absence of the compound and detecting binding between the VEGF-C and the binding partner in the presence and absence of the compound, wherein differential binding in the presence and absence of the compound identifies the compound as a modulator of binding between the VEGF-C and the binding partner; and wherein the binding partner is selected from the group consisting of:

(i) a polypeptide comprising a VEGFR-3 extracellular domain; and

(ii) a polypeptide comprising a VEGFR-2 extracellular domain; and

(c) identifying the selectivity of the modulator compound in view of the binding detected in steps (a) and (b).

Step (a) of the above embodiment involves contacting a neuropilin composition with a VEGF-C composition as described previously. Step (b) of the outlined method involves contacting a VEGF-C composition as described in step (a) with a composition comprising a VEGF-C binding partner in the presence and in the absence of the same compound. The VEGF-C binding partner is selected from the group consisting of: (i) a polypeptide comprising a VEGFR-3 extracellular domain; and (ii) a polypeptide comprising a VEGFR-2 extracellular domain. Thus, the above-described embodiment involves measuring selectivity of a modulator of VEGF-C/neuropilin binding in relation to VEGF-C binding to its receptors, VEGFR-2 and VEGFR-3. The VEGF-C binding partner chosen is preferably of vertebrate origin, more preferably mammalian, still more preferably primate, and still more preferably human. And, while it will be apparent that the assay will likely give its best results if the functional portion of the chosen VEGF-C binding partner is identical in amino acid sequence to the native VEGF-C binding partner, it will be apparent that the invention can still be practiced if variations have been introduced in the VEGF-C binding partner sequence that do not eliminate its VEGF-C binding properties. Use of variant sequences with at least 90%, 95%, 96%, 97%, 98%, or 99% amino acid identity is specifically contemplated. Any technique for detecting intermolecular binding may be employed. For example, one or both of the binding partner or the VEGF-C may comprise a label, such as a radioisotope, a fluorophore, a fluorescing protein (e.g., natural or synthetic green fluorescent proteins), a die, an enzyme or substrate, or the like. Such labels facilitate detection with standard laboratory machinery and techniques.

In one variation, the binding partner composition comprises a cell that expresses the binding partner naturally or recombinantly on its surface. In this situation, it will often be possible to detect VEGF-C binding indirectly, e.g., by detecting or measuring a VEGF-C binding-induced physiological change in the cell. Such possible changes include phosphorylation of the associated VEGFR; cell chemotaxis; cell growth, changes in cellular morphology; ionic fluxes, or the like.

Step (c) of the outlined method involves identifying the selectivity of the modulator compound on the basis of increased or decreased binding in steps (a) and (b). A compound that is a selective modulator causes significant differential binding in either step (a) or step (b), but does not cause significant differential binding in both steps (a) and (b). A non-specific modulator causes significant differential binding in both steps (a) and (b).

In still another embodiment, the invention provides a method for screening for selectivity of a modulator of neuropilin biological activity, comprising steps of:

a) contacting a neuropilin composition with a VEGF-C composition in the presence and in the absence of a compound and detecting binding between the neuropilin and the VEGF-C in the presence and absence of the compound, wherein differential binding in the presence and absence of the compound identifies the compound as a modulator of binding between the neuropilin and the VEGF-C;

b) contacting a neuropilin composition with a composition comprising a neuropilin binding partner in the presence and in the absence of the compound and detecting binding between the neuropilin and the binding partner in the presence and absence of the compound, wherein differential binding in the presence and absence of the compound identifies the compound as a modulator of binding between the neuropilin and the binding partner; and wherein the binding partner is selected from the group consisting of:

(i) a polypeptide comprising an amino acid sequence of a semaphorin 3 polypeptide,

(ii) a polypeptide comprising a VEGF-A amino acid sequence, a VEGF-B amino acid sequence, a VEGF-D amino acid sequence, a PlGF-2 amino acid sequence, a VEGFR-1 amino acid sequence, a VEGFR-2 amino acid sequence, a VEGFR-3 amino acid sequence; and

(iii) a polypeptide comprising an amino acid sequence of a plexin polypeptide

d) identifying the selectivity of the modulator compound in view of the binding detected in steps (a) and (b).

Step (a) of the above embodiment involves contacting a neuropilin composition with a VEGF-C composition as described previously. Step (b) of the outlined method involves contacting a neuropilin composition as described in step (a) with a composition comprising a neuropilin binding partner in the presence and in the absence of a compound. The neuropilin binding partner comprises any protein other than VEGF-C that the neuropilin binds. Exemplary binding partners include the following polypeptides: a polypeptide comprising the amino acid sequence of a semaphorin 3 family member polypeptide; a polypeptide comprising a VEGF-A amino acid sequence, a polypeptide comprising a VEGF-B amino acid sequence, a polypeptide comprising a VEGF-D amino acid sequence, a polypeptide comprising a PlGF-2 amino acid sequence, a polypeptide comprising a VEGFR-1 amino acid sequence, a polypeptide comprising a VEGFR-2 amino acid sequence, a polypeptide comprising a VEGFR-3 amino acid sequence; and a polypeptide comprising the amino acid sequence of a plexin family member. The binding partners chosen are preferably of vertebrate origin, more preferably mammalian, still more preferably primate, and still more preferably human. And, while it will be apparent that the assay will likely give its best results if the functional portion of the chosen neuropilin binding partner is identical in amino acid sequence to the native sequence, it will be apparent that the invention can still be practiced if variations have been introduced in the native sequence that do not eliminate its neuropilin binding properties. Use of variant sequences with at least 90%, 95%, 96%, 97%, 98%, or 99% amino acid identity is specifically contemplated.

The above-described method includes detecting binding between the neuropilin composition and the binding partner in the presence and absence of the compound. Any technique for detecting intermolecular binding may be employed. For example, one or both of the binding partner or the neuropilin may comprise a label, such as a radioisotope, a fluorophore, a fluorescing protein (e.g., natural or synthetic green fluorescent proteins), a dye, an enzyme or substrate, or the like. Such labels facilitate detection with standard laboratory machinery and techniques.

Step (c) of the outlined method involves identifying the selectivity of the modulator compound on the basis of increased or decreased binding in steps (a) and (b), and having the characteristics of a selective modulator compound as described previously.

In an additional embodiment, the invention provides a method of screening for modulators of binding between a neuropilin growth factor receptor and a VEGFR-3 polypeptide comprising steps of:

a) contacting a neuropilin composition with a VEGFR-3 composition in the presence and in the absence of a putative modulator compound;

b) detecting binding between the neuropilin and the VEGFR-3 in the presence and absence of the putative modulator compound; and

c) identifying a modulator compound based on a decrease or increase in binding between the neuropilin composition and the VEGFR-3 composition in the presence of the putative modulator compound, as compared to binding in the absence of the putative modulator compound.

Step (a) of the aforementioned method involves contacting a neuropilin composition as described with a VEGFR-3 composition in the presence and absence of a putative modulator compound. The neuropilin composition contemplated is described previously. A VEGFR-3 composition comprises a member selected from the group consisting of (i) a composition comprising a purified polypeptide that comprises an entire VEGFR-3 protein or that comprises a VEGFR-3 fragment that binds the neuropilin; (ii) a composition containing phospholipid membranes that contain VEGFR-3 polypeptides on their surface; (iii) a living cell recombinantly modified to express increased amounts of a VEGFR-3 on its surface; and (iv) any isolated cell or tissue that naturally expresses the VEGFR-3 on its surface. For certain assay formats, it may be desirable to bind the VEGFR-3 molecule of interest (e.g., a polypeptide comprising a VEGFR-3 extracellular domain fragment) to a solid support such as a bead or assay plate well. “VEGFR-3 composition” is intended to include such structures as well. Likewise, fusion proteins are contemplated. For other assay formats, soluble VEGFR-3 peptides may be preferred. In one preferred variation, the VEGFR-3 receptor composition comprises a VEGFR-3 receptor fragment fused to an immunoglobulin Fc fragment.

Step (b) of the above method involves detecting binding between the neuropilin composition and the VEGFR-3 composition in the presence and absence of the compound. Any technique for detecting intermolecular binding may be employed. For example, one or both of neuropilin/VEGFR-3 may comprise a label, such as a radioisotope, a fluorophore, a fluorescing protein (e.g., natural or synthetic green fluorescent proteins), a dye, an enzyme or substrate, or the like. Such labels facilitate detection with standard laboratory machinery and techniques.

Generally, more attractive modulators are those that will activate or inhibit neuropilin-VEGFR-3 binding at lower concentrations, thereby permitting use of the modulators in a pharmaceutical composition at lower effective doses.

In another embodiment, the invention provides for a method for screening for selectivity of a modulator of VEGFR-3 biological activity, comprising steps of:

a) contacting a VEGFR-3 composition with a neuropilin composition in the presence and in the absence of a compound and detecting binding between the VEGFR-3 and the neuropilin in the presence and absence of the compound, wherein differential binding in the presence and absence of the compound identifies the compound as a modulator of binding between the VEGFR-3 and the neuropilin;

b) contacting a VEGFR-3 composition with a composition comprising a VEGFR-3 binding partner in the presence and in the absence of a compound and detecting binding between the VEGFR-3 and the binding partner in the presence and absence of the compound, wherein differential binding in the presence and absence of the compound identifies the compound as a modulator of binding between the VEGFR-3 and the binding partner; and wherein the binding partner is selected from the group consisting of:

(i) a polypeptide comprising a VEGF-C polypeptide; and

(ii) a polypeptide comprising a VEGF-D polypeptide; and

c) identifying the selectivity of the modulator compound in view of the binding detected in steps (a) and (b).

A selective modulator causes significant differential binding in either step (a) or step (b), but does not cause significant differential binding in both steps (a) and (b).

It will be apparent that the foregoing selectivity screens represent only a portion of the specific selectivity screens of the present invention, because the neuropilins, VEGF-C, VEGF-D, and VEGFR-3 all have multiple binding partners, creating a number of permutations for selectivity screens. Any selectivity screen that involves looking at one of the following interactions: (i) neuropilin-1/VEGF-C; (ii) neuropilin-1/VEGF-D; (iii) neuropilin-2/VEGF-C; (iv) neuropilin-2/VEGF-D; (v) neuropilin-1/VEGFR-3; and (vi) neuropilin-2/VEGFR3; together with at least one other interaction (e.g., a known interaction of one of these molecules, or a second interaction from the foregoing list) is specifically contemplated as part of the present invention.

Likewise, all of the screens for modulators and the selectivity screens optionally comprising one or both of the following steps: (1) making a modulator composition by formulating a chosen modulator in a pharmaceutically acceptable carrier; and (2) administering the modulator so formulated to an animal or human and determining the effect of the modulator. Preferably, the animal or human has a disease or condition involving one of the foregoing molecular interactions, and the animal or human is monitored to determine the effect of the modulator on the disease or condition, which, hopefully, is ameliorated or cured.

The discovery of neuropilin-2 and neuropilin-1 binding to VEGF-C molecules provides new and useful materials and methods for investigating biological processes involved in many currently known disease states. For example, the invention provides for a method of modulating growth, migration, or proliferation of cells in a mammalian organism, comprising a step of:

(a) identifying a mammalian organism having cells that express a neuropilin receptor; and

(b) administering to said mammalian organism a composition, said composition comprising a neuropilin polypeptide or fragment thereof that binds to a VEGF-C polypeptide;

wherein the composition is administered in an amount effective to modulate growth, migration, or proliferation of cells that express neuropilin in the mammalian organism. Administration of soluble forms of the neuropilin is preferred.

Preferably, the mammalian organism is human. Also, the cells preferably comprise vascular endothelial cells, especially cells of lymphatic origin, such as human microvascular endothelial cells (HMVEC) and human cutaneous fat pad microvascular cells (HUCEC). In a highly preferred embodiment, the organism has a disease characterized by aberrant growth, migration, or proliferation of endothelial cells. The administration of the agent beneficially alters the aberrant growth, migration, or proliferation, e.g., by correcting it, or reducing its severity, or reducing its deleterious symptoms or effects.

For example, in one variation, the animal has a cancer, especially a cancerous tumor characterized by vasculature containing neuropilin-expressing endothelial cells. A composition is selected that will decrease growth, migration, or proliferation of the cells, and thereby retard the growth of the tumor by preventing growth of new vasculature. In such circumstances, one may wish to administer agents that inhibit other endothelial growth factor/receptor interactions, such as inhibitors of the VEGF-family of ligands; endostatins; inhibitory angiopoietins, or the like. Exemplary inhibitors include antibody substances specific for the growth factors or their ligands. The invention further contemplates treating lymphangioamas, lymphangiosarcomas, and metastatic tumors, which exhibit VEGFR-3 expressing vascular endothelial cells or VEGFR-3 expressing lymphatic endothelial cells. In one embodiment, administration of a composition that inhibits the interaction of VEGFR-3 with its ligand diminishes or abolishes lymphangiogenesis and retards the spread of cancerous cells. In an additional embodiment, administration of a composition that stimulates the interaction of VEGFR-3 with its ligand enhances lymphangiogenesis and speeds wound healing.

Further contemplated is a method of modulating growth, migration, or proliferation of cells in a mammalian organism, comprising steps of:

(a) identifying a mammalian organism having cells that express a neuropilin receptor; and

(b) administering to said mammalian organism a composition, said composition comprising a bispecific antibody specific for the neuropilin receptor and for a VEGF-C polypeptide, wherein the composition is administered in an amount effective to modulate growth, migration, or proliferation of cells that express the neuropilin receptor in the mammalian organism. In an alternative embodiment, the bispecific antibody is specific for the neuropilin receptor and for a VEGFR-3 polypeptide.

In one embodiment, the invention provides a bispecific antibody which specifically binds a neuropilin receptor and a VEGF-C polypeptide. Alternatively, the invention provides a bispecific antibody which specifically binds to the neuropilin receptor and a VEGFR-3 polypeptide.

In another embodiment, the invention can also be used to inhibit neural degeneration in the central nervous system. Development of scars surrounding neuronal injury in either the peripheral and more specifically the central nervous system has been associated with constitutive expression of the semaphorin ligands. Also, upregulation of Sema3F, a primary ligand for the neuropilin-2 receptor, has been detected in the brains of Alzheimer's patients. The present invention provides for a means to alter the semaphorin-neuropilin interactions using VEGF-C compositions that specifically interfere with semaphorin activity in the nervous system.

For example, the invention provides for a method of modulating aberrant growth, or neuronal scarring in a mammalian organism, comprising a step of:

(a) identifying a mammalian organism having neuronal cells that express a neuropilin receptor; and

(b) administering to said mammalian organism a composition, said composition comprising a VEGF-C polypeptide or fragment thereof that binds to the neuropilin receptor;

wherein the composition is administered in an amount effective to reduce neuronal scarring in cells that express neuropilin in the mammalian organism.

Other conditions to treat include inflammatory diseases (e.g., Rheumatoid arthritis, chronic wounds and atherosclerosis).

Similarly, the invention provides a polypeptide comprising a fragment of VEGF-C that binds to a neuropilin receptor, for use in the manufacture of a medicament for the treatment of diseases characterized by aberrant growth, migration, or proliferation of cells that express a neuropilin receptor.

Likewise, the invention provides a polypeptide comprising a fragment of a neuropilin that binds to a VEGF-C, for use in the manufacture of a medicament for the treatment of diseases characterized by aberrant growth, migration, or proliferation of cells that express a neuropilin receptor. Soluble forms of the neuropilin, lacking the transmembrane domain, are preferred. The invention also provides for a polypeptide comprising a fragment of a neuropilin receptor that binds to a VEGFR-3 polypeptide, for use in the manufacture of a medicament for the treatment of diseases characterized by aberrant growth, migration, or proliferation of cells that express a VEGFR-3 polypeptide.

With respect to aspects of the invention that involve administration of protein agents to mammals, a related aspect of the invention comprises gene therapy whereby a gene encoding the protein of interest is administered in a manner to effect expression of the protein of interest in the animal. For example, the gene of interest is attached to a suitable promoter to promote expression of the protein in the target cell of interest, and is delivered in any gene therapy vector capable of delivering the gene to the cell, including adenovirus vectors, adeno-associated virus vectors, liposomes, naked DNA transfer, and others.

Additional features and variations of the invention will be apparent to those skilled in the art from the entirety of this application, and all such features are intended as aspects of the invention.

Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 depicts the construction of the neuropilin-2 IgG fusion protein a17 and a22 expression vectors.

### DETAILED DESCRIPTION OF THE INVENTION

The present invention is based, in part, on the discovery of novel interaction between proteins that have previously been characterized in the literature, but whose interactions were not previously appreciated. A number of the molecules are explicitly set forth with annotations to the Genbank database or to a Sequence Listing appended hereto, but it will be appreciated that sequences for species homologous (“orthologs”) are also easily retrieved from databases and/or isolated from natural sources. Thus, the following table and description should be considered exemplary and not limiting.

A. Molecules of interest to the present invention.*

| Molecule | Genbank Accession # | SEQ ID NO. |
|---|---|---|
| Neuropilin-1 | NM003873 | 1 and 2 |
| Soluble Neuropilin-1, s11 | AF280547 | |
| Neuropilin-2 [a(17)] | NM003872 | 3 and 4 |
| a(0) | AF022859 | |
| a(17) | AF022860 | |
| b(0) | AF280544 | |
| b(5) | AF280545 | |
| Soluble Neuropilin-2, s9 | AF280546 | |
| Murine neuropilin-1 | D50086 | 5 and 6 |
| Murine neuropilin-2 | | |
| a(0) | AF022854 | |
| a(5) | AF022861 | |
| a(17) | AF022855 | 7 and 8 |
| a(22) | AF022856 | |
| b(0) | AF022857 | |
| b(5) | AF022858 | |
| Semaphorin 3A | NM006080 | 9 and 10 |
| Semaphorin 3B | NM004636 | 11 and 12 |
| Semaphorin 3C | NM006379 | 13 and 14 |
| Semaphorin 3E | NM012431 | 15 and 16 |
| Semaphorin 3F | NM004186 | 17 and 18 |
| VEGF-A | Q16889 | 19 and 20 |
| VEGF165 | M32977 | |
| VEGF-B | U48801 | 21 and 22 |
| VEGF-C | X94216 | 23 and 24 |
| VEGF-D | AJ000185 | 25 and 26 |
| VEGF-E | S67522 | |
| P1GF | NM002632 | 27 and 28 |
| VEGFR-1 | X51602 | |
| VEGFR-2 | L04947 | 29 and 30 |
| VEGFR-3 | X68203 | 31 and 32 |
| Plexin-A1 | X87832 | |
| Plexin-A2 | NM025179 | |
| PDGF-A,-B,-C | NM002607; NM002608; NM016205 | |
| PDGFR-A,-B | NM006206; NM002609 | |

*All Sequences of Human origin unless otherwise noted.

The Neuropilin Family

The neuropilin-1 and neuropilin-2 genes span over 120 and 112 kb, respectively, and are comprised of 17 exons, five of which are identical in size in both genes, suggesting genetic duplication of these genes (Rossignol et al, Genomics 70:211-22. 2000). Several splice variants of the neuropilins have been isolated to date, the functional significance of which is currently under investigation.

Isoforms of NRP-2, designated NRP2a and NRP2b, were first isolated from the mouse genome (Chen et al, Neuron 19:547-59. 1997). In mouse, NRP2a isoforms contain insertions of 0, 5, 17, or 22 (5+17) amino acids after amino acid 809 of NRP-2 and are named NRP2a(0) (Genbank Accession No. AF022854)(SEQ ID NO. 7 and 8), NRP2a(5) (Genbank Accession No. AF022861), NRP2a(17) (Genbank Accession No. AF022855), and NRP2a(22)(Genbank Accession No. AF022856), respectively. Only two human NRP2a isoforms homologous to the mouse variants NRP2a(17) (Genbank Accession No. AF022860) (SEQ ID NO. 3 and 4) and NRP2a(22), have been elucidated. The human a(22) isoform contains a five amino acid insertion, sequence GENFK, after amino acid 808 in NRP2a(17). Tissue analysis of brain, heart, lung, kidney liver and placenta shows that the a(17) isoform is more abundant in all of these sites.

The human NRP2b isoforms appear to express an additional exon, designated exon 16b, not present in either NRP2a or NRP-1. Two human NRP2b isoforms homologous to mouse NRP2b(0) (Genbank Accession No. AF022857) and NRP2b(5) (Genbank Accession No. AF022858) have been identified which contain either a 0 or 5 amino acid insert (GENFK) after amino acid 808 in NRP2b(0) (Rossignol et al., Genomics 70:211-22. 2000). Tissue distribution analysis demonstrates a higher expression of human NRP2b(0) (Genbank Accession No. AF280544) over NRP2b(5) (Genbank Accession No. AF280545) in adult brain, heart, lung, kidney, liver, and placenta. The NRP2a and NRP2b isoforms demonstrate divergence in their C terminal end, after amino acid 808 of NRP2 which is in the linker region between the c domain and the transmembrane domain. This differential splicing may lead to the difference seen in tissue expression of the two isoforms, where NRP2a is expressed more abundantly in the placenta, liver, and lung with only detectable levels of NRP2b, while NRP2b is found in skeletal muscle where NRP2a expression is low. Both isoforms are expressed in heart and small intestine.

In addition to genetic isoforms of the neuropilins, truncated soluble forms of the proteins have also been cloned (Gagnon et al, Proc. Natl. Acad. Sci USA 97:2573-78 2000; Rossignol et al, Genomics 70:211-22. 2000). Naturally occurring truncated forms of the NRP-1 protein, s11NRP1 (Genbank Accession No. AF280547) and s12NRP1, have been cloned, that encode 704 and 644 amino acid neuropilin-1, respectively, and contain the a and b domains but not the c domain. The s12NRP1 variant is generated by pre-mRNA processing in intron 12. The s11NRP1 truncation occurs after amino acid 621 and lacks the 20 amino acids encoded by exon 12, but contains coding sequence found within intron 11 that gives it 83 novel amino acids at the C-terminus. This intron derived sequence does not contain any homology to known proteins.

A natural, soluble form of NRP-2 has also been identified which encodes a 555 amino acid protein containing the a domains, b1 domain, and part of the b2 domain, lacking the last 48 amino acids of this region. The truncation occurs after amino acid 547 within intron 9, thus the protein has been named s9NRP2 (Genbank Accession No. AF2805446), and adds 8 novel amino acids derived from the intron cleavage (VGCSVWRPL) at the C-terminus. Gagnon et al (Proc. Natl. Acad. Sci USA 97:2573-78. 2000) report that soluble neuropilin-1 isoform s12NRP1 is capable of binding VEGF165 equivalent to the full length protein, but acts as an antagonist of VEGF165 binding, inhibiting VEGF165 activity and showing anti-tumor properties in a rat prostate carcinoma model.

The PDGF/VEGF Family

The PDGF/VEGF family of growth factors includes at least the following members: PDGF-A (see e.g., GenBank Acc. No. X06374), PDGF-B (see e.g., GenBank Acc. No. M12783), VEGF (see e.g., GenBank Acc. No. Q16889 referred to herein for clarity as VEGF-A or by particular isoform), PlGF (see e.g., GenBank Acc. No. X54936 placental growth factor), VEGF-B (see e.g., GenBank Acc. No. U48801; also known as VEGF-related factor (VRF)), VEGF-C (see e.g., GenBank Acc. No. X94216; also known as VEGF related protein (VRP or VEGF-2)), VEGF-D (also known as c-fos-induced growth factor (FIGF); see e.g., Genbank Acc. No. AJ000185), VEGF-E (also known as NZ7 VEGF or OV NZ7; see e.g., GenBank Acc. No. S67522), NZ2 VEGF (also known as OV NZ2; see e.g., GenBank Acc. No. S67520), D1701 VEGF-like protein (see e.g., GenBank Acc. No. AF106020; Meyer et al., EMBO J 18:363-374), and NZ10 VEGF-like protein (described in International Patent Application PCT/US99/25869) [Stacker and Achen, Growth Factors 17: 1-11 (1999); Neufeld et al., FASEB J 13:9-22 (1999); Ferrara, J Mol Med 77:527-543 (1999)]. The PDGF/VEGF family proteins are predominantly secreted glycoproteins that form either disulfide-linked or non-covalently bound homo- or heterodimers whose subunits are arranged in an anti-parallel manner [Stacker and Achen, Growth Factors 17:1-11 (1999); Muller et al., Structure 5:1325-1338 (1997)].

The VEGF subfamily is composed of PDGF/VEGF members which share a VEGF homology domain (VHD) characterized by the sequence: C-X(22-24)-P-[PSR]-C-V-X(3)-R-C-[GSTA]-G-C-C-X(6)-C-X(32-41)-C.

VEGF-A was originally purified from several sources on the basis of its mitogenic activity toward endothelial cells, and also by its ability to induce microvascular permeability, hence it is also called vascular permeability factor (VPF). VEGF-A has subsequently been shown to induce a number of biological processes including the mobilization of intracellular calcium, the induction of plasminogen activator and plasminogen activator inhibitor-1 synthesis, promotion of monocyte migration in vitro, induction of anti-apoptotic protein expression in human endothelial cells, induction of fenestrations in endothelial cells, promotion of cell adhesion molecule expression in endothelial cells and induction of nitric oxide mediated vasodilation and hypotension [Ferrara, J Mol Med 77: 527-543 (1999); Neufeld et al., FASEB J 13: 9-22 (1999); Zachary, Intl J Biochem Cell Bio 30: 1169-1174 (1998)].

VEGF-A is a secreted, disulfide-linked homodimeric glycoprotein composed of 23 kD subunits. Five human VEGF-A isoforms of 121, 145, 165, 189 or 206 amino acids in length (VEGF₁₂₁₋₂₀₆), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparin-sulfate proteoglycans, which behave as low affinity receptors for VEGF-A. VEGF₁₂₁ does not bind to either heparin or heparin-sulfate; VEGF₁₄₅ and VEGF₁₆₅ (GenBank Acc. No. M32977) are both capable of binding to heparin; and VEGF₁₈₉ and VEGF₂₀₆ show the strongest affinity for heparin and heparin-sulfates. VEGF₁₂₁, VEGF₁₄₅, and VEGF₁₆₅ are secreted in a soluble form, although most of VEGF₁₆₅ is confined to cell surface and extracellular matrix proteoglycans, whereas VEGF₁₈₉ and VEGF₂₀₆ remain associated with extracellular matrix. Both VEGF₁₈₉ and VEGF₂₀₆ can be released by treatment with heparin or heparinase, indicating that these isoforms are bound to extracellular matrix via proteoglycans. Cell-bound VEGF₁₈₉ can also be cleaved by proteases such as plasmin, resulting in release of an active soluble VEGF₁₁₀. Most tissues that express VEGF are observed to express several VEGF isoforms simultaneously, although VEGF₁₂₁ and VEGF₁₆₅ are the predominant forms, whereas VEGF₂₀₆ is rarely detected [Ferrara, J Mol Med 77:527-543 (1999)]. VEGF₁₄₅ differs in that it is primarily expressed in cells derived from reproductive organs [Neufeld et al., FASEB J 13:9-22 (1999)].

The pattern of VEGF-A expression suggests its involvement in the development and maintenance of the normal vascular system, and in angiogenesis associated with tumor growth and other pathological conditions such as rheumatoid arthritis. VEGF-A is expressed in embryonic tissues associated with the developing vascular system, and is secreted by numerous tumor cell lines. Analysis of mice in which VEGF-A was knocked out by targeted gene disruption indicate that VEGF-A is critical for survival, and that the development of the cardiovascular system is highly sensitive to VEGF-A concentration gradients. Mice lacking a single copy of VEGF-A die between day 11 and 12 of gestation. These embryos show impaired growth and several developmental abnormalities including defects in the developing cardiovasculature. VEGF-A is also required post-natally for growth, organ development, regulation of growth plate morphogenesis and endochondral bone formation. The requirement for VEGF-A decreases with age, especially after the fourth postnatal week. In mature animals, VEGF-A is required primarily for active angiogenesis in processes such as wound healing and the development of the corpus luteum. [Neufeld et al., FASEB J 13:9-22 (1999); Ferrara, J Mol Med 77:527-543 (1999)]. VEGF-A expression is influenced primarily by hypoxia and a number of hormones and cytokines including epidermal growth factor (EGF), TGF-β, and various interleukins. Regulation occurs transcriptionally and also post-transcriptionally such as by increased mRNA stability [Ferrara, J Mol Med 77:527-543 (1999)].

PlGF, a second member of the VEGF subfamily, is generally a poor stimulator of angiogenesis and endothelial cell proliferation in comparison to VEGF-A, and the in vivo role of PlGF is not well understood. Three isoforms of PlGF produced by alternative mRNA splicing have been described [Hauser et al., Growth Factors 9:259-268 (1993); Maglione et al., Oncogene 8:925-931 (1993)]. PlGF forms both disulfide-linked homodimers and heterodimers with VEGF-A. The PlGF-VEGF-A heterodimers are more effective at inducing endothelial cell proliferation and angiogenesis than PlGF homodimers. PlGF is primarily expressed in the placenta, and is also co-expressed with VEGF-A during early embryogenesis in the trophoblastic giant cells of the parietal yolk sac [Stacker and Achen, Growth Factors 17:1-11 (1999)].

VEGF-B, described in detail in International Patent Publication No. WO 96/26736 and U.S. Pat. Nos. 5,840,693 and 5,607,918, incorporated herein by reference, shares approximately 44% amino acid identity with VEGF-A. Although the biological functions of VEGF-B in vivo remain incompletely understood, it has been shown to have angiogenic properties, and may also be involved in cell adhesion and migration, and in regulating the degradation of extracellular matrix. It is expressed as two isoforms of 167 and 186 amino acid residues generated by alternative splicing. VEGF-B₁₆₇ is associated with the cell surface or extracellular matrix via a heparin-binding domain, whereas VEGF-B₁₈₆ is secreted. Both VEGF-B₁₆₇ and VEGF-B₁₈₆ can form disulfide-linked homodimers or heterodimers with VEGF-A. The association to the cell surface of VEGF₁₆₅-VEGF-B₁₆₇ heterodimers appears to be determined by the VEGF-B component, suggesting that heterodimerization may be important for sequestering VEGF-A. VEGF-B is expressed primarily in embryonic and adult cardiac and skeletal muscle tissues [Joukov et al., J Cell Physiol 173:211-215 (1997); Stacker and Achen, Growth Factors 17:1-11 (1999)]. Mice lacking VEGF-B survive but have smaller hearts, dysfunctional coronary vasculature, and exhibit impaired recovery from cardiac ischemia [Bellomo et al., Circ Res 2000;E29-E35].

A fourth member of the VEGF subfamily, VEGF-C, comprises a VHD that is approximately 30% identical at the amino acid level to VEGF-A. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. Prepro-VEGF-C undergoes extensive proteolytic maturation involving the successive cleavage of a signal peptide, the C-terminal pro-peptide, and the N-terminal pro-peptide. Secreted VEGF-C protein consists of a non-covalently-linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. [Joukov et al., EMBO J., 16:(13):3898-3911 (1997).] It has also been demonstrated that a mutant VEGF-C, in which a single cysteine at position 156 is either substituted by another amino acid or deleted, loses the ability to bind VEGFR-2 but remains capable of binding and activating VEGFR-3 [U.S. Pat. No. 6,130,071 and International Patent Publication No. WO 98/33917]. In mouse embryos, VEGF-C mRNA is expressed primarily in the allantois, jugular area, and the metanephros. [Joukov et al., J Cell Physiol 173:211-215 (1997)]. VEGF-C is involved in the regulation of lymphatic angiogenesis: when VEGF-C was overexpressed in the skin of transgenic mice, a hyperplastic lymphatic vessel network was observed, suggesting that VEGF-C induces lymphatic growth [Jeltsch et al., Science, 276:1423-1425 (1997)]. Continued expression of VEGF-C in the adult also indicates a role in maintenance of differentiated lymphatic endothelium [Ferrara, J Mol Med 77:527-543 (1999)]. VEGF-C also shows angiogenic properties: it can stimulate migration of bovine capillary endothelial (BCE) cells in collagen and promote growth of human endothelial cells [see, e.g., U.S. Pat. No. 6,245,530; U.S. Pat. No. 6,221,839; and International Patent Publication No. WO 98/33917, incorporated herein by reference].

The prepro-VEGF-C polypeptide is processed in multiple stages to produce a mature and most active VEGF-C polypeptide of about 21-23 kD (as assessed by SDS-PAGE under reducing conditions). Such processing includes cleavage of a signal peptide (SEQ ID NO: 24, residues 1-31); cleavage of a carboxyl-terminal peptide (corresponding approximately to amino acids 228-419 of SEQ ID NO: 24 and having a pattern of spaced cysteine residues reminiscent of a Balbiani ring 3 protein (BR3P) sequence [Dignam et al., Gene, 88:133-40 (1990); Paulsson et al., J. Mol. Biol., 211:331-49 (1990)]) to produce a partially-processed form of about 29 kD; and cleavage (apparently extracellularly) of an amino-terminal peptide (corresponding approximately to amino acids 32-103 of SEQ ID NO: 24) to produced a fully-processed mature form of about 21-23 kD. Experimental evidence demonstrates that partially-processed forms of VEGF-C (e.g., the 29 kD form) are able to bind the Flt4 (VEGFR-3) receptor, whereas high affinity binding to VEGFR-2 occurs only with the fully processed forms of VEGF-C. It appears that VEGF-C polypeptides naturally associate as non-disulfide linked dimers.

Moreover, it has been demonstrated that amino acids 103-227 of SEQ ID NO: 24 are not all critical for maintaining VEGF-C functions. A polypeptide consisting of amino acids 113-213 (and lacking residues 103-112 and 214-227) of SEQ ID NO: 24 retains the ability to bind and stimulate VEGF-C receptors, and it is expected that a polypeptide spanning from about residue 131 to about residue 211 will retain VEGF-C biological activity. The cysteine residue at position 156 has been shown to be important for VEGFR-2 binding ability. However, VEGF-C ΔC156 polypeptides (i.e., analogs that lack this cysteine due to deletion or substitution) remain potent activators of VEGFR-3. The cysteine at position 165 of SEQ ID NO: 24 is essential for binding either receptor, whereas analogs lacking the cysteines at positions 83 or 137 compete with native VEGF-C for binding with both receptors and stimulate both receptors.

VEGF-D is structurally and functionally most closely related to VEGF-C [see U.S. Pat. No. 6,235,713 and International Patent Publ. No. WO 98/07832, incorporated herein by reference]. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D (SEQ ID NO: 26) optionally linked to the affinity tag peptide FLAG®, or other sequences.

The prepro-VEGF-D polypeptide has a putative signal peptide of 21 amino acids and is apparently proteolytically processed in a manner analogous to the processing of prepro-VEGF-C. A “recombinantly matured” VEGF-D lacking residues 1-92 and 202-354 of SEQ ID NO: 26 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201 of SEQ ID NO: 26. The guidance provided above for introducing function-preserving modifications into VEGF-C polypeptides is also suitable for introducing function-preserving modifications into VEGF-D polypeptides.

Four additional members of the VEGF subfamily have been identified in poxviruses, which infect humans, sheep and goats. The orf virus-encoded VEGF-E and NZ2 VEGF are potent mitogens and permeability enhancing factors. Both show approximately 25% amino acid identity to mammalian VEGF-A, and are expressed as disulfide-linked homodimers. Infection by these viruses is characterized by pustular dermatitis which may involve endothelial cell proliferation and vascular permeability induced by these viral VEGF proteins. [Ferrara, J Mol Med 77:527-543 (1999); Stacker and Achen, Growth Factors 17:1-11 (1999)]. VEGF-like proteins have also been identified from two additional strains of the orf virus, D1701 [GenBank Acc. No. AF106020; described in Meyer et al., EMBO J 18:363-374 (1999)] and NZ10 [described in International Patent Application PCT/US99/25869, incorporated herein by reference]. These viral VEGF-like proteins have been shown to bind VEGFR-2 present on host endothelium, and this binding is important for development of infection and viral induction of angiogenesis [Meyer et al., EMBO J 18:363-374 (1999); International Patent Application PCT/US99/25869].

PDGF/VEGF Receptors

Seven cell surface receptors that interact with PDGF/VEGF family members have been identified. These include PDGFR-α (see e.g., GenBank Acc. No. NM006206), PDGFR-β (see e.g., GenBank Acc. No. NM002609), VEGFR-1/Flt-1 (fms-like tyrosine kinase-1; GenBank Acc. No. X51602; De Vries et al., Science 255:989-991 (1992)); VEGFR-2/KDR/Flk-1 (kinase insert domain containing receptor/fetal liver kinase-1; GenBank Acc. Nos. X59397 (Flk-1) and L04947 (KDR); Terman et al., Biochem Biophys Res Comm 187:1579-1586 (1992); Matthews et al., Proc Natl Acad Sci USA 88:9026-9030 (1991)); VEGFR-3/Flt4 (fms-like tyrosine kinase 4; U.S. Pat. Nos. 5,776,755 and GenBank Acc. No. X68203 and S66407; Pajusola et al., Oncogene 9:3545-3555 (1994)), neuropilin-1 (Gen Bank Acc. No. NM003873), and neuropilin-2 (Gen Bank Acc. No. NM003872). The two PDGF receptors mediate signaling of PDGFs as described above. VEGF121, VEGF165, VEGF-B, PlGF-1 and PlGF-2 bind VEGF-R1; VEGF121, VEGF145, VEGF165, VEGF-C, VEGF-D, VEGF-E, and NZ2 VEGF bind VEGF-R2; VEGF-C and VEGF-D bind VEGFR-3; VEGF165, VEGF-B, PlGF-2, and NZ2 VEGF bind neuropilin-1; and VEGF165, and VEGF145 bind neuropilin-2.[Neufeld et al., FASEB J 13:9-22 (1999); Stacker and Achen, Growth Factors 17:1-11 (1999); Ortega et al., Fron Biosci 4:141-152 (1999); Zachary, Intl J Biochem Cell Bio 30:1169-1174 (1998); Petrova et al., Exp Cell Res 253:117-130 (1999); Gluzman-Poltorak et al., J. Biol. Chem. 275:18040-45 (2000)].

The PDGF receptors are protein tyrosine kinase receptors (PTKs) that contain five immunoglobulin-like loops in their extracellular domains. VEGFR-1, VEGFR-2, and VEGFR-3 comprise a subgroup of the PDGF subfamily of PTKs, distinguished by the presence of seven Ig domains in their extracellular domain and a split kinase domain in the cytoplasmic region. Both neuropilin-1 and neuropilin-2 are non-PTK VEGF receptors, with short cytoplasmic tails not currently known to possess downstream signaling capacity.

Several of the VEGF receptors are expressed as more than one isoform. A soluble isoform of VEGFR-1 lacking the seventh Ig-like loop, transmembrane domain, and the cytoplasmic region is expressed in human umbilical vein endothelial cells. This VEGFR-1 isoform binds VEGF-A with high affinity and is capable of preventing VEGF-A-induced mitogenic responses [Ferrara, J Mol Med 77:527-543 (1999); Zachary, Intl J Biochem Cell Bio 30:1169-1174 (1998)]. A C-terminal truncated from of VEGFR-2 has also been reported [Zachary, Intl J Biochem Cell Bio 30:1169-1174 (1998)]. In humans, there are two isoforms of the VEGFR-3 protein which differ in the length of their C-terminal ends. Studies suggest that the longer isoform is responsible for most of the biological properties of VEGFR-3.

The expression of VEGFR-1 occurs mainly in vascular endothelial cells, although some may be present on monocytes, trophoblast cells, and renal mesangial cells [Neufeld et al., FASEB J 13:9-22 (1999)]. High levels of VEGFR-1 mRNA are also detected in adult organs, suggesting that VEGFR-1 has a function in quiescent endothelium of mature vessels not related to cell growth. VEGFR-1 −/− mice die in utero between day 8.5 and 9.5. Although endothelial cells developed in these animals, the formation of functional blood vessels was severely impaired, suggesting that VEGFR-1 may be involved in cell-cell or cell-matrix interactions associated with cell migration. Recently, it has been demonstrated that mice expressing a mutated VEGFR-1 in which only the tyrosine kinase domain was missing show normal angiogenesis and survival, suggesting that the signaling capability of VEGFR-1 is not essential. [Neufeld et al., FASEB J 13:9-22 (1999); Ferrara, J Mol Med 77:527-543 (1999)].

VEGFR-2 expression is similar to that of VEGFR-1 in that it is broadly expressed in the vascular endothelium, but it is also present in hematopoietic stem cells, megakaryocytes, and retinal progenitor cells [Neufeld et al., FASEB J 13:9-22 (1999)]. Although the expression pattern of VEGFR-1 and VEGFR-2 overlap extensively, evidence suggests that, in most cell types, VEGFR-2 is the major receptor through which most of the VEGFs exert their biological activities. Examination of mouse embryos deficient in VEGFR-2 further indicate that this receptor is required for both endothelial cell differentiation and the development of hematopoietic cells [Joukov et al., J Cell Physiol 173:211-215 (1997)].

VEGFR-3 is expressed broadly in endothelial cells during early embryogenesis. During later stages of development, the expression of VEGFR-3 becomes restricted to developing lymphatic vessels [Kaipainen, A., et al., Proc. Natl. Acad. Sci. USA, 92: 3566-3570 (1995)]. In adults, the lymphatic endothelia and some high endothelial venules express VEGFR-3, and increased expression occurs in lymphatic sinuses in metastatic lymph nodes and in lymphangioma. VEGFR-3 is also expressed in a subset of CD34+ hematopoictic cells which may mediate the myelopoietic activity of VEGF-C demonstrated by overexpression studies [WO 98/33917]. Targeted disruption of the VEGFR-3 gene in mouse embryos leads to failure of the remodeling of the primary vascular network, and death after embryonic day 9.5 [Dumont et al., Science, 282: 946-949 (1998)]. These studies suggest an essential role for VEGFR-3 in the development of the embryonic vasculature, and also during lymphangiogenesis.

Structural analyses of the VEGF receptors indicate that the VEGF-A binding site on VEGFR-1 and VEGFR-2 is located in the second and third Ig-like loops. Similarly, the VEGF-C and VEGF-D binding sites on VEGFR-2 and VEGFR-3 are also contained within the second Ig-loop [Taipale et al., Curr Top Microbiol Immunol 237:85-96 (1999)]. The second Ig-like loop also confers ligand specificity as shown by domain swapping experiments [Ferrara, J Mol Med 77:527-543 (1999)]. Receptor-ligand studies indicate that dimers formed by the VEGF family proteins are capable of binding two VEGF receptor molecules, thereby dimerizing VEGF receptors. The fourth Ig-like loop on VEGFR-1, and also possibly on VEGFR-2, acts as the receptor dimerization domain that links two receptor molecules upon binding of the receptors to a ligand dimer [Ferrara, J Mol Med 77:527-543 (1999)]. Although the regions of VEGF-A that bind VEGFR-1 and VEGFR-2 overlap to a large extent, studies have revealed two separate domains within VEGF-A that interact with either VEGFR-1 or VEGFR-2, as well as specific amino acid residues within these domains that are critical for ligand-receptor interactions. Mutations within either VEGF receptor-specific domain that specifically prevent binding to one particular VEGF receptor have also been recovered [Neufeld et al., FASEB J 13:9-22 (1999)].

VEGFR-1 and VEGFR-2 are structurally similar, share common ligands (VEGF121 and VEGF165), and exhibit similar expression patterns during development. However, the signals mediated through VEGFR-1 and VEGFR-2 by the same ligand appear to be slightly different. VEGFR-2 has been shown to undergo autophosphorylation in response to VEGF-A, but phosphorylation of VEGFR-1 under identical conditions was barely detectable. VEGFR-2 mediated signals cause striking changes in the morphology, actin reorganization, and membrane ruffling of porcine aortic endothelial cells recombinantly overexpressing this receptor. In these cells, VEGFR-2 also mediated ligand-induced chemotaxis and mitogenicity; whereas VEGFR-1-transfected cells lacked mitogenic responses to VEGF-A. Mutations in VEGF-A that disrupt binding to VEGFR-2 fail to induce proliferation of endothelial cells, whereas VEGF-A mutants that are deficient in binding VEGFR-1 are still capable of promoting endothelial proliferation. Similarly, VEGF stimulation of cells expressing only VEGFR-2 leads to a mitogenic response whereas comparable stimulation of cells expressing only VEGFR-1 also results in cell migration, but does not induce cell proliferation. In addition, phosphoproteins co-precipitating with VEGFR-1 and VEGFR-2 are distinct, suggesting that different signaling molecules interact with receptor-specific intracellular sequences.

The emerging hypothesis is that the primary function of VEGFR-1 in angiogenesis may be to negatively regulate the activity of VEGF-A by binding it and thus preventing its interaction with VEGFR-2, whereas VEGFR-2 is thought to be the main transducer of VEGF-A signals in endothelial cells. In support of this hypothesis, mice deficient in VEGFR-1 die as embryos while mice expressing a VEGFR-1 receptor capable of binding VEGF-A but lacking the tyrosine kinase domain survive and do not exhibit abnormal embryonic development or angiogenesis. In addition, analyses of VEGF-A mutants that bind only VEGFR-2 show that they retain the ability to induce mitogenic responses in endothelial cells. However, VEGF-mediated migration of monocytes is dependent on VEGFR-1, indicating that signaling through this receptor is important for at least one biological function. In addition, the ability of VEGF-A to prevent the maturation of dendritic cells is also associated with VEGFR-1 signaling, suggesting that VEGFR-1 may function in cell types other than endothelial cells. [Ferrara, J Mol Med 77:527-543 (1999); Zachary, Intl J Biochem Cell Bio 30:1169-1174 (1998)].

With respect to the neuropilins or other polypeptides used to practice the invention, it will be understood that native sequences will usually be most preferred. By “native sequences” is meant sequences encoded by naturally occurring polynucleotides, including but not limited to prepro-peptides, pro-peptides, and partially and fully proteolytically processed polypeptides. As described above, many of the polypeptides have splice variants that exist, e.g., due to alternative RNA processing, and such splice variants comprise native sequences. For purposes described herein, fragments of the forgoing that retain the binding properties of interest also shall be considered native sequences. Moreover, modifications can be made to most protein sequences without destroying the activity of interest of the protein, especially conservative amino acid substitutions, and proteins so modified are also suitable for practice of the invention. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

Moreover, deletion and addition of amino acids is often possible without destroying a desired activity. With respect to the present invention, where binding activity is of particular interest and the ability of molecules to activate or inhibit receptor tyrosine kinases upon binding is of special interest, binding assays and tyrosine phophorylation assays are available to determine whether a particular ligand or ligand variant (a) binds and (b) stimulates or inhibits RTK activity.

Two manners for defining genera of polypeptide variants include percent amino acid identity to a native polypeptide (e.g., 80, 85, 90, 91, 92, 93, 94, 95, 96, 97, 98, or 99% identity preferred), or the ability of encoding-polynucleotides to hybridize to each other under specified conditions. One exemplary set of conditions is as follows: hybridization at 42° C. in 50% formamide, 5×SSC, 20 mM Na.PO4, pH 6.8; and washing in 1×SSC at 55° C. for 30 minutes. Formula for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel, et al. (Eds.), Protocols in Molecular Biology, John Wiley & Sons (1994), pp.6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

B. Gene Therapy

While much of the application, including the examples, are written in the context of protein-protein interactions and protein administration, it should be clear that genetic manipulations to achieve modulation of protein expression or activity is specifically contemplated. For example, where administration of proteins is contemplated, administration of a gene therapy vector to cause the protein of interest to be produced in vivo also is contemplated. Where inhibition of proteins is contemplated (e.g., through use of antibodies or small molecule inhibitors), inhibition of protein expression in vivo by genetic techniques, such as knock-out techniques or anti-sense therapy, is contemplated.

Any suitable vector may be used to introduce a transgene of interest into an animal. Exemplary vectors that have been described in the literature include replication-deficient retroviral vectors, including but not limited to lentivirus vectors [Kim et al., J. Virol., 72(1): 811-816 (1998); Kingsman & Johnson, Scrip Magazine, October, 1998, pp. 43-46.]; adeno-associated viral vectors [Gnatenko et al., J. Investig. Med., 45: 87-98 (1997)]; adenoviral vectors [See, e.g., U.S. Pat. No. 5,792,453; Quantin et al., Proc. Natl. Acad. Sci. USA, 89: 2581-2584 (1992); Stratford-Perricadet et al., J. Clin. Invest., 90: 626-630 (1992); and Rosenfeld et al., Cell, 68: 143-155 (1992)]; Lipofectin-mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in the entirety. Replication-deficient adenoviral vectors and adeno-associated viral vectors constitute preferred embodiments.

In embodiments employing a viral vector, preferred polynucleotides include a suitable promoter and polyadenylation sequence to promote expression in the target tissue of interest. For many applications of the present invention, suitable promoters/enhancers for mammalian cell expression include, e.g., cytomegalovirus promoter/enhancer [Lehner et al., J. Clin. Microbiol., 29:2494-2502 (1991); Boshart et al., Cell, 41:521-530 (1985)]; Rous sarcoma virus promoter [Davis et al., Hum. Gene Ther., 4:151 (1993)]; or simian virus 40 promoter.

Anti-sense polynucleotides are polynucleotides which recognize and hybridize to polynucleotides encoding a protein of interest and can therefore inhibit transcription or translation of the protein. Full length and fragment anti-sense polynucleotides may be employed. Commercial software is available to optimize antisense sequence selection and also to compare selected sequences to known genomic sequences to help ensure uniqueness/specificity for a chosen gene. Such uniqueness can be further confirmed by hybridization analyses. Antisense nucleic acids (preferably 10 to 20 base pair oligonucleotides) are introduced into cells (e.g., by a viral vector or colloidal dispersion system such as a liposome). The antisense nucleic acid binds to the target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end.

Genetic control can also be achieved through the design of novel transcription factors for modulating expression of the gene of interest in native cells and animals. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular target sequence of the present invention facilitates the engineering of zinc finger proteins specific for the target sequence using known methods such as a combination of structure-based modeling and screening of phage display libraries [Segal et al., (1999) Proc Natl Acad Sci USA 96:2758-2763; Liu et al., (1997) Proc Natl Acad Sci USA 94:5525-30; Greisman and Pabo (1997) Science 275:657-61; Choo et al., (1997) J Mol Biol 273:525-32]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., (1999) Proc Natl Acad Sci USA 96:2758-2763]. The artificial zinc finger repeats, designed based on target sequences, are fused to activation or repression domains to promote or suppress gene expression [Liu et al., (1997) Proc Natl Acad Sci USA 94:5525-30]. Alternatively, the zinc finger domains can be fused to the TATA box-binding factor (TBP) with varying lengths of linker region between the zinc finger peptide and the TBP to create either transcriptional activators or repressors [Kim et al., (1997) Proc Natl Acad Sci USA 94:3616-3620]. Such proteins, and polynucleotides that encode them, have utility for modulating expression in vivo in both native cells, animals and humans. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., (1999) Proc Natl Acad Sci USA 96:9521-6; Wu et al., (1995) Proc Natl Acad Sci USA 92:344-348].

C. Antibodies

Antibodies are useful for modulating Neuropilin-VEGF-C interactions due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for polypeptides of interest to the invention, especially neuropilins, VEGF receptors, and VEGF-C and VEGF-D proteins. Preferred antibodies are human antibodies which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind the polypeptide of interest exclusively (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known and routinely practiced in the art.

Bispecific antibodies are monoclonal, preferably human or humanized, antibodies that have binding specificities for at least two different antigens. In the present case, one of the binding specificities is for NRP-2, the other one is for an NRP-2 binding partner, and preferably for a cell-surface protein or receptor or receptor subunit, such as VEGFR-3.

In one embodiment, a bispecific antibody which binds to both NRP-2 and VEGFR-3 is used to modulate the growth, migration or proliferation of cells that results from the interaction of VEGF-C with VEGFR-3. For example, the bispecific antibody is administered to an individual having tumors characterized by lymphatic metastasis or other types of tumors expressing both VEGF-C and VEGFR-3, and NRP-2. The bispecific antibody which binds both NRP-2 and VEGFR-3 blocks the binding of VEGF-C to VEGFR-3, thereby interfereing with VEGF-C mediated lymphangiogenesis and slowing the progression of tumor metastatsis. In another embodiment, the same procedure is carried out with a bispecific antibody which binds to NRP-2 and VEGF-C, wherein administration of said antibody sequesters soluble VEGF-C and prevents its binding to VEGFR-3, effectively acting as an inhibitor of VEGF-C mediated signaling through VEGFR-3.

Bispecific antibodies are produced, isolated, and tested using standard procedures that have been described in the literature. See, e.g., Pluckthun & Pack, Immunotechnology, 3:83-105 (1997); Carter et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal et al., J. Hematotherapy, 4: 377-382 (1995); Segal et al., Immunobiology, 185: 390-402 (1992); and Bolhuis et al., Cancer Immunol. Immunother., 34: 1-8 (1991), all of which are incorporated herein by reference in their entireties.

The term “bispecific antibody” refers to a single, divalent antibody which has two different antigen binding sites (variable regions). As described below, the bispecific binding agents are generally made of antibodies, antibody fragments, or analogs of antibodies containing at least one complementarity determining region derived from an antibody variable region. These may be conventional bispecific antibodies, which can be manufactured in a variety of ways (Holliger, P. and Winter G. Current Opinion Biotechnol. 4, 446-449 (1993)), e.g. prepared chemically, using hybrid hybridomas, via linking the coding sequence of such a bispecific antibody into a vector and producing the recombinant peptide or by phage display. The bispecific antibodies may also be any bispecific antibody fragments.

In one method, bispecific antibodies fragments are constructed by converting whole antibodies into (monospecific) F(ab′)₂ molecules by proteolysis, splitting these fragments into the Fab′ molecules and recombine Fab′ molecules with different specificity to bispecific F(ab′)₂ molecules (see, for example, U.S. Pat. No. 5,798,229).

A bispecific antibody can be generated by enzymatic conversion of two different monoclonal antibodies, each comprising two identical L (light chain)-H (heavy chain) half molecules and linked by one or more disulfide bonds, into two F(ab′)₂ molecules, splitting each F(ab′)2 molecule under reducing conditions into the Fab′ thiols, derivatizing one of these Fab′ molecules of each antibody with a thiol activating agent and combining an activated Fab′ molecule bearing NRP-2 specificity with a non-activated Fab′ molecule bearing an NRP-2 binding partner specificity or vice versa in order to obtain the desired bispecific antibody F(ab′)₂ fragment.

As enzymes suitable for the conversion of an antibody into its F(ab′)₂ molecules, pepsin and papain may be used. In some cases, trypsin or bromelin are suitable. The conversion of the disulfide bonds into the free SH-groups (Fab′ molecules) may be performed by reducing compounds, such as dithiothreitol (DTT), mercaptoethanol, and mercaptoethylamine. Thiol activating agents according to the invention which prevent the recombination of the thiol half-molecules, are 5,5′-dithiobis(2-nitrobenzoic acid) (DTNB), 2,2′-dipyridinedisulfide, 4,4′-dipyridinedisulfide or tetrathionate/sodium sulfite (see also Raso et al., Cancer Res., 42:457 (1982), and references incorporated therein).

The treatment with the thiol-activating agent is generally performed only with one of the two Fab′ fragments. Principally, it makes no difference which one of the two Fab′ molecules is converted into the activated Fab′ fragment (e.g., Fab′-TNB). Generally, however, the Fab′ fragment being more labile is modified with the thiol-activating agent. In the present case, the fragments bearing the anti-tumor specificity are slightly more labile, and, therefore, preferably used in the process. The conjugation of the activated Fab′ derivative with the free hinge-SH groups of the second Fab′ molecule to generate the bivalent F(ab′)₂ antibody occurs spontaneously at temperatures between 0° and 30° C. The yield of purified F(ab′)₂ antibody is 20-40% (starting from the whole antibodies).

Another method for producing bispecific antibodies is by the fusion of two hybridomas to form a hybrid hybridoma. As used herein, the term “hybrid hybridoma” is used to describe the productive fusion of two B cell hybridomas. Using now standard techniques, two antibody producing hybridomas are fused to give daughter cells, and those cells that have maintained the expression of both sets of clonotype immunoglobulin genes are then selected.

To identify the bispecific antibody standard methods such as ELISA are used wherein the wells of microtiter plates are coated with a reagent that specifically interacts with one of the parent hybridoma antibodies and that lacks cross-reactivity with both antibodies. In addition, FACS, immunofluorescence staining, idiotype specific antibodies, antigen binding competition assays, and other methods common in the art of antibody characterization may be used in conjunction with the present invention to identify preferred hybrid hybridomas.

Bispecific molecules of this invention can also be prepared by conjugating a gene encoding a binding specificity for NRP-2 to a gene encoding at least the binding region of an antibody chain which recognizes a binding partner of NRP-2 such as VEGF-C or VEGFR-3. This construct is transfected into a host cell (such as a myeloma) which constitutively expresses the corresponding heavy or light chain, thereby enabling the reconstitution of a bispecific, single-chain antibody, two-chain antibody (or single chain or two-chain fragment thereof such as Fab) having a binding specificity for NRP-2 and for a NRP-2 binding partner. Construction and cloning of such a gene construct can be performed by standard procedures.

Bispecific antibodies are also generated via phage display screening methods using the so-called hierarchical dual combinatorial approach as disclosed in WO 92/01047 in which an individual colony containing either an H or L chain clone is used to infect a complete library of clones encoding the other chain (L or H) and the resulting two-chain specific binding member is selected in accordance with phage display techniques such as those described therein. This technique is also disclosed in Marks et al, (Bio/Technology, 1992, 10:779-783).

The bispecific antibody fragments of the invention can be administered to human patients for therapy. Thus, in one embodiment the bispecific antibody is provided with a pharmaceutical formulation comprising as active ingredient at least one bispecific antibody fragment as defined above, associated with one or more pharmaceutically acceptable carrier, excipient or diluent. In another embodiment, the compound further comprises an anti-neoplastic or cytotoxic agent conjugated to the bispecific antibody.

Recombinant antibody fragments, e.g. scFvs, can also be engineered to assemble into stable multimeric oligomers of high binding avidity and specificity to different target antigens. Such diabodies (dimers), triabodies (trimers) or tetrabodies (tetramers) are well known within the art and have been described in the literature, see e.g. Kortt et al., Biomol Eng. Oct. 15, 2001;18(3):95-108 and Todorovska et al., J Immunol Methods. Feb. 1, 2001;248(1-2):47-66.

Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

D. Dosing

Some methods of the invention include a step of polypeptide administration to a human or animal. Polypeptides may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises (in addition to the polynucleotide or vector) a pharmaceutically-acceptable carrier solution such as water, saline, phosphate-buffered saline, glucose, or other carriers conventionally used to deliver therapeutics or imaging agents.

The “administering” that is performed according to the present invention may be performed using any medically-accepted means for introducing a therapeutic directly or indirectly into a mammalian subject, including but not limited to injections (e.g., intravenous, intramuscular, subcutaneous, or catheter); oral ingestion; intranasal or topical administration; and the like. For some cardiovascular diseases a preferred route of administration is intravascular, such as by intravenous, intra-arterial, or intracoronary arterial injection. In one embodiment, administering the composition is performed at the site of a lesion or affected tissue needing treatment by direct injection into the lesion site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the lesion.

The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

Polypeptides for administration may be formulated with uptake or absorption enhancers to increase their efficacy. Such enhancer include for example, salicylate, glycocholate/linoleate, glycholate, aprotinin, bacitracin, SDS caprate and the like. See, e.g., Fix (J. Pharm. Sci., 85(12) 1282-1285, 1996) and Oliyai and Stella (Ann. Rev. Pharmacol. Toxicol., 32:521-544, 1993).

The amounts of peptides in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day. These concentrations may be administered as a single dosage form or as multiple doses. Standard dose-response studies, first in animal models and then in clinical testing, reveal optimal dosages for particular disease states and patient populations.

It will also be apparent that dosing should be modified if traditional therapeutics are administered in combination with therapeutics of the invention. For example, treatment of cancer using traditional chemotherapeutic agents or radiation, in combination with methods of the invention, is contemplated.

E. Kits

As an additional aspect, the invention includes kits which comprise one or more compounds or compositions of the invention packaged in a manner which facilitates their use to practice methods of the invention. In a simplest embodiment, such a kit includes a compound or composition described herein as useful for practice of a method of the invention (e.g., polynucleotides or polypeptides for administration to a person or for use in screening assays), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration or for practicing a screening assay.

Additional aspects and details of the invention will be apparent from the following examples, which are intended to be illustrative rather than limiting.

### EXAMPLE 1

### VEGF-C Isoforms Bind to Neuropilin-2 and Neuropilin-1

The following experiments demonstrated that VEGF-C isoforms interact with the neuropilin family members, neuropilin-2 and neuropilin-1.

A. Materials

To investigate the binding of neuropilin-2 to VEGF-C the following constructs were either made or purchased from commercial sources:

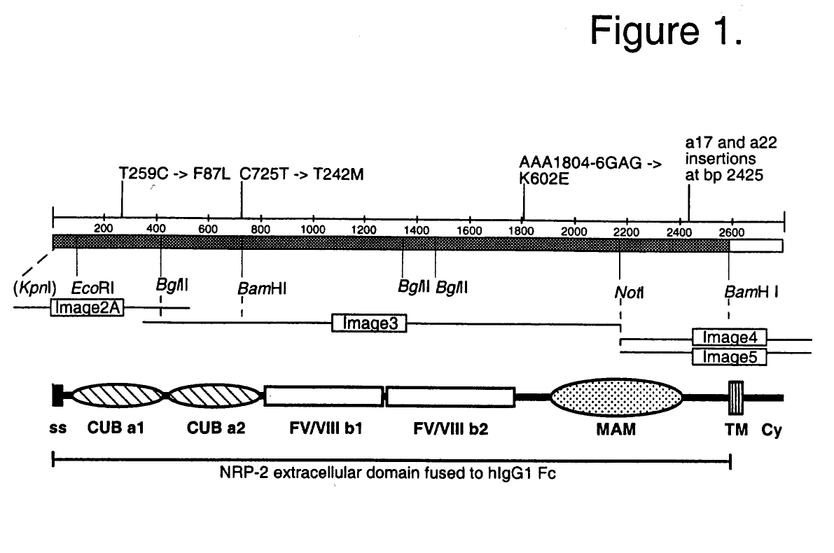

a) Cloning of the NRP-2/IgG expression vector. The extracellular domain of hNRP-2 was cloned into the plgplus vector in frame with the human IgG1 Fc tail as follows. Full-length NRP-2 cDNA (SEQ ID NO. 3) was assembled from several IMAGE Consortium cDNA Clones (Incyte Genomics) (FIG. 1A). The Image clones used are marked as 2A (GenBank Acc. No AA621145; Clone ID 1046499), 3 (AA931763; 1564852), 4 (AA127691; 490311), and 5 (AW296186; 2728688); these clones were confirmed by sequencing. Image clones 4 and 5 differ due to alternative splicing, coding for a17 and a22 isoforms, respectively. The BamHI-NotI fragment from the image clone 3 was first cloned into the pcDNA3.1z+ vector (Invitrogen), and fragments KpnI-Bgl II from clone 2A and Bgl II-BamHI from clone 3 were then added to obtain the 5′ region (bp 1-2188). NotI-BamHI fragments from clones 4 and 5 were separately transferred into the pIgplus vector, and the KpnI-NotI fragment from the pcDNA3.1z+ vector was then inserted to obtain the expression vector coding for the extracellular domain of the hNRP-2/IgG fusion protein (SEQ ID NO. 3, positions 1 to 2577). The NRP-2 inserts in the resulting vectors were sequenced. The Image clone 3 codes for one amino acid different from the GenBank Sequence (AAA 1804-1806 GAG|K602E). However, the amino acid sequence in the Image clone 3 is identical to the original sequence published by Chen et al. (Chen et al., Neuron, 19:547. 1997).

b) a VEGFR-3-Fc construct, in which an extracellular domain portion of VEGFR-3 comprising the first three immunoglobulin-like domains (SEQ ID NO. 32, amino acids 1 to 329) was fused to the Fc portion of human IgG1 [see Makinen et al., Nat Med., 7:199-205 (2001)]. Full length VEGFR-3 cDNA and amino acid sequences are set forth in SEQ. ID NOS: 31 and 32.

c) a NRP-1-Fc construct, in which an extracellular domain portion of murine NRP-1 (base pairs 248-2914 of SEQ. ID NO: 5) was fused to the Fc portion of human IgG1 (Makinen et al, J. Biol.Chem 274:21217-222. 1999); and

d) the expression vectors, in pREP7 backbone, encoding either VEGF165 (Genbank Accession No. M32997) or full-length VEGF-C (SEQ. ID NO: 24), have been described recently (Olofsson et al., Proc. Natl. Acad. Sci. USA 93: 2576-81. 1996; and Joukov et al., EMBO J. 15: 290-298. 1996).

B. Co-immunoprecipitation of VEGF-C with NRP-2

The NRP-2, NRP-1, and VEGFR-3 pIgplus fusion constructs were transfected into 293T cells using the FUGENETM6 transfection reagent (Roche Molecular Biochemicals). The cells were grown in Dulbecco's modified Eagle's medium supplemented with 10% fetal calf serum (Gibco BRL), glutamine, and antibiotics. The media was replaced 48 h after transfection by DMEM containing 0.2% BSA and collected after 20 h.

For growth factor production, 293EBNA cells were transfected with expression vectors coding for VEGF₁₆₅, prepro-VEGF-C, or empty vector (Mock). 36 h after transfection, the cells were first incubated in methionine and cysteine free MEM (Gibco BRL) for 45 min, metabolically labeled in the same medium supplemented with 100 millicurie [mCi]/ml Pro-mix [35S] (Amersham) for 6-7 h (1 mCi=37 kBq) containing radiolabelled methionine and cysteine.

For immunoprecipitation controls, 1 ml of the labeled medium was incubated with either MAB 293 monoclonal anti-VEGF-Ab (R&D Systems), or rabbit antiserum 882 against VEGF-C (Joukov et al., EMBO J. 16:3898-3911. 1997) for 2 h, with rotation, at +4° C. Protein A-Sepharose (Pharmacia) was then added, and incubated overnight. The immunoprecipitates were washed two times with ice-cold PBS-0.5% Tween 20, heated in Laemmli sample buffer, and electrophoresed in 15% SDS PAGE. The gel was dried and exposed to Kodak Biomax MR film.

For binding experiments, the labeled supernatants from the Mock- or VEGF-C transfected cells were first immunoprecipitated with VEGF antibodies (R & D Systems) for depletion of endogenous VEGF. 4 ml of hNRP-2 a17-IgG or 1 ml of VEGFR-3-IgG or NRP-1-IgG fusion protein containing media were incubated with 1 ml of growth factor containing media (Mock, VEGF or VEGF-C) in binding buffer (0.5% BSA, 0.02% Tween 20) for 2 h, Protein A-Sepharose was added, and incubated overnight. The samples were then washed once with ice-cold binding buffer and three times with PBS and subjected to 15% SDS PAGE. The radiolabeled VEGF-C polypeptide was detected via chemiluminescence (ECL).

Results show that both the 29 kD and 21-23 kD isoforms of VEGF-C bind to NRP-2 while only the 29 kD form binds to NRP-1. VEGFR-3 binding to VEGF-C was used as a positive control for VEGF-C binding in the assay. It has been shown previously that heparin strongly increases VEGF binding to NRP-2 (Gluzman-Poltorak et al., J. Biol.Chem. 275: 18040-045. 2000). Addition of heparin to the assay mixture illustrates that VEGF₁₆₅ binding to NRP-2 is heparin dependent while VEGF₁₆₅ binding to NRP-1 is independent of heparin binding, and the presence of heparin has no effect on VEGF-C binding to any of its receptors.

C. Cell-based Assay Using Cells that Naturally Express Neuropilin Receptors.

The preceding experiment can be modified by substituting cells that naturally express a neuropilin receptor (especially NRP-2) for the transfected 293EBNA cells. Use of primary cultures of neuronal cells expressing neuropilin receptors is specifically contemplated, e.g., cultured cerebellar granule cells derived from embryos. Additionally, NRP-receptor-specific antibodies can be employed to identify other cells (e.g., cells involved in the vasculature), such as human microvascular endothelial cells (HMVEC), human cutaneous fat pad microvascular cells (HUCEC) that express NRP receptors.

### EXAMPLE 2

### Neuropilin-2 Interacts With VEGFR-3

Recent results indicate that NRP-1 is a co-receptor for VEGF₁₆₅ binding, forming a complex with VEGFR-2, which results in enhanced VEGF₁₆₅ signaling through VEGFR-2, over VEGF₁₆₅ binding to VEGFR-2 alone, thereby enhancing the biological responses to this ligand (Soker et al., Cell 92: 735-45. 1998). A similar phenomenon may apply to VEGF-C signaling via possible VEGFR-3/NRP-2 receptor complexes.

A. Binding Assay

The NRP-2(a22) expression vector was cloned as described in Example 1 (FIG. 1B) with the addition of a detectable tag on the 3′ end. For 3′ end construction, the Not I-Bam HI fragment (clone 5) was then constructed by PCR, introducing the V5 tag (GKPIPNPLLGLDST) (SEQ ID NO:33) and a stop codon to the 3′ terminus. To obtain the expression vector coding for the full-length hNRP-2(a22) protein, this 3′ end was then transferred into the vector containing the 5′ fragment. The resulting clone was referred to as V5 NRP-2.

To determine the interaction of VEGFR-3 with NRP-2, 10 cm plates of human embryonic kidney cells (293T or 293EBNA) were transfected with the V5 NRP-2 construct or VEGFR-3 using 6 μl of FUGENE TM6 (Roche Molecular Biochemicals, Indianapolis, Ind.) and 2 μg DNA. The cells were grown in Dulbecco's modified Eagle's medium supplemented with 10% fetal calf serum (Gibco BRL), glutamine, and antibiotics. For Mock transfections, 2 μg of empty vector was used. For single receptor transfections, the VEGFR-3-myc/pcDNA3.1 (Karkkainen et al, Nat. Genet. 25:153-59. 2000) or NRP-2(a22)/pcDNA3.1z+and empty vector were used in a one to one ratio. The VEGFR-3/NRP-2 co-transfections were also made in a one to one ratio. After 24 h, the 293EBNA cells were starved overnight, and stimulated for 10 min using 300 ng/ml ΔNΔCVEGF-C (produced in P. pastoris; (Joukov et al. EMBOJ. 16: 3898-3911. 1997)). The cells were then washed twice with ice-cold PBS containing vanadate (100 μM) and PMSF (100 μM), and lysed in dimerization lysis buffer (20 mM HEPES pH 7.5,150 mM NaCl,10% glycerol, 1% Triton X-100,2 mM MgCl2, 2 mM CaCl2 , 10 μg/ml bovine serum albumin (BSA)) containing 2 mM vanadate, 1 mM PMSF, 0.07 U/ml aprotinin, and 4 μg/ml leupeptin. The lysates were cleared by centrifugation for 10 min at 19,000 g, and incubated with antibodies for VEGFR-3 (9d9F;(Jussila et al., Cancer Res. 58: 1599-1604. 1998)), or V5 (Invitrogen) for 5 h at +4° C. The immunocomplexes were then incubated with protein A-Sepharose (Pharmacia) overnight at +4° C., the immunoprecipitates were washed four times with dimerization lysis buffer without BSA, and the samples subjected to 7.5% SDS-PAGE in reducing conditions. The proteins were transferred to a Protran nitrocellulose filter (Schleicher & Schuell) using semi-dry transfer apparatus. After blocking with 5% non-fat milk powder in TBS-T buffer (10 mM Tris pH 7.5,150 mM NaCl, 0.1% Tween 20), the filters were incubated with the V5 antibodies, followed by HRP-conjugated rabbit-anti-mouse immunoglobulins (Dako), and visualized using enhanced chemiluminescence (ECL).

Co-immunoprecipitation of VEGFR-3 and NRP-2 constructs transfected into 293T cells demonstrates that NRP-2 interacts with VEGFR-3 when co-expressed in the same cell. Immunoprecipitation after the addition of VEGF-C to the cell culture media shows that the NRP-2/VEGFR-3 interaction is not dependent on the presence of the VEGF-C ligand, implying that these receptors may associate naturally in vivo without the presence of VEGF-C. This finding may have tremendous implications on the binding and activity of VEGF-C during angiogenesis. VEGF-C, an integral molecule in promoting growth and development of the lymphatic vasculature, is also highly involved in the metastasis of cancerous cells through the lymph system and apparently the neovascularization of at least some solid tumors (see International Patent Publication No. WO 00/21560). The novel interaction between neuropilins and VEGF-C provides for a means to specifically block this lymphatic growth into solid tumors by inhibiting lymphatic cell migration as a result of VEGF-C binding to VEGFR-3. Neuropilins-1 and-2 are the only VEGF receptors at the surface of some tumor cells, indicating the binding of VEGF to neuropilins is relevant to tumor growth (Soker et al, Cell 92: 735-45. 1998) and that VEGF-C binding to neuropilin-2 may be a means to specifically target tumor metastasis through the lymphatic system.

### EXAMPLE 3

### Inhibition of VEGF-C Binding to VEGFR-3 by Neuropilins

The binding affinity between VEGF-C and neuropilin receptor molecules provides therapeutic indications for modulators of VEGF-C-induced VEGFR-3 receptor signaling, in order to modulate, i.e. stimulate or inhibit, VEGF-receptor-mediated biological processes. The following examples are designed to provide proof of this therapeutic concept.

A. In vitro Cell-free Assay

To demonstrate the inhibitory effects of neuropilin-1-Fc and neuropilin-2-Fc against VEGF-C stimulation, a label, e.g. a biotin molecule, is fused with the VEGF-C protein and first incubated with neuropilin-1-Fc, neuropilin-2-Fc, VEGFR-2 Fc or VEGFR-3-Fc at various molar ratios, and then applied on microtiter plates pre-coated with 1 microgram/ml of VEGFR-3 or VEGFR-2. After blocking with 1% BSA/PBS-T, fresh, labeled VEGF-C protein or the VEGF-C/receptor-Fc mixture above is applied on the microtiter plates overnight at 4 degrees Centigrade. Thereafter, the plates are washed with PBS-T, and 1:1000 of avidin-HRP will be added. Bound VEGF-C protein is detected by addition of the ABTS substrate (KPL). The bound labeled VEGF-C is analyzed in the presence and absence of the soluble neuropilins or soluble VEGFRs and the percent inhibition of binding assessed, as well as the effects the neuropilins have on binding to either VEGFR-2 or VEGFR-3 coated microtiter plates. In a related variation, this assay is carried out substituting VEGF-D for VEGF-C.

B. In vitro Cell-based Assay

VEGF-C is used as described above to contact cells that naturally or recombinantly express NRP-2 and VEGFR-3 receptors on their surface. By way of example, 293EBNA or 293T cells recombinantly modified to transiently or stably express neuropilins and VEGFR-3 as outlined above are employed. Several native endothelial cell types express both receptors and can also be employed, including but not limited to, human microvascular endothelial cells (HMEC) and human cutaneous fat pad microvascular cells (HUCEC).

For assessment of autophosphorylation of VEGFR-3, 293T or 293EBNA human embryonic kidney cells grown in Dulbecco's modified Eagle's medium (DMEM) supplemented with 10% fetal calf serum (GIBCO BRL), glutamine and antibiotics, are transfected using the FUGENE TM6 transfection reagent (Roche Molecular Biochemicals) with plasmid DNAs encoding the receptor constructs (VEGFR-3 or VEGFR-3-myc tag and/or neuropilin-V5 tag,) or an empty pcDNA3.1z+ vector (Invitrogen). For stimulation assay, the 293EBNA cell monolayers are starved overnight (36 hours after transfection) in serum-free medium containing 0.2% BSA. The 293EBNA cells are then stimulated with 300 ng/ml recombinant DNDC VEGF-C (Joukov et al., EMBO J. 16:3898-3911. 1997) for 10 min at +37° C., in the presence or absence of neuropilin-Fc to determine inhibition of VEGF-C/VEGFR-3 binding. The cells are then washed twice with cold phosphate buffered saline (PBS) containing 2 mM vanadate and 2 mM phenylmethylsulfonyl fluoride (PMSF), and lysed into PLCLB buffer (150 mM NaCl, 5% glycerol, 1% Triton X-100, 1.5 M MgCl2, and 50 mM Hepes, pH 7.5) containing 2 mM Vanadate, 2 mM PMSF, 0.07 U/ml Aprotinin, and 4 mg/ml leupeptin. The lysates are centrifuged for 10 min at 19 000 g, and incubated with the supernatants for 2 h on ice with 2 μg/ml of monoclonal anti-VEGFR-3 antibodies (9D9f9) (Jussila et al., Cancer Res. 58:1599-1604. 1998), or alternatively with antibodies against the specific tag epitopes (1.1 mg/ml of anti-V5 antibodies (Invitrogen) or 5 μg/ml anti-Myc antibodies (BabCO). The immunocomplexes are incubated with protein A sepharose (Pharmacia) for 45 min with rotation at +4° C. and the sepharose beads washed three times with cold PLCLB buffer (2 mM vanadate, 2 mM PMSF). The bound polypeptides are separated by 7.5% SDS-PAGE and transferred to a Protran nitrocellulose filter (Schleicher & Schuell) using semi-dry transfer apparatus. After blocking with 5% BSA in TBS-T buffer (10 mM Tris pH 7.5, 150 mM NaCl, 0.1% Tween 20), the filters are stained with the phosphotyrosine-specific primary antibodies (Upstate Biotechnology), followed by biotinylated goat-anti-mouse immunoglobulins (Dako) and Biotin-Streptavidin HRP complex (Amersham) Phosphotyrosine-specific bands are visualized by enhanced chemiluminescence (ECL). To analyze the samples for the presence of VEGFR-3, the filters are stripped for 30 min at +55° C. in 100 mM 2-mercaptoethanol, 2% SDS, 62.5 mM Tris-HCl pH 6.7 with occasional agitation, and stained with 9D9f9 antibodies and HRP conjugated rabbit-anti-mouse immunoglobulins (Dako) for antigen detection. Reduced VEGFR-3 autophosphorylation is indicative of successful neuropilin-Fc-mediated inhibition of VEGF-C/VEGFR3 binding.

VEGF-C protein naturally secreted into media conditioned by a PC-3 prostatic adenocarcinoma cell line (ATCC CRL 1435) in serum-free Ham's F-12 Nutrient mixture (GIBCO) (containing 7% fetal calf serum (FCS)) (U.S. Pat. No. 6,221,839) can be used to activate VEGFR3 expressing cells in vitro. For in vitro assay purposes, cells can be reseeded and grown in this medium, which is subsequently changed to serum-free medium. As shown in a previous experiment, pretreatment of the concentrated PC-3 conditioned medium with 50 microliters of VEGFR-3 extracellular domain coupled to CNBr-activated sepharose CL-4B (Pharmacia; about 1 mg of VEGFR-3EC domain/ml sepharose resin) completely abolished VEGFR-3 tyrosine phosphorylation (U.S. Pat. No. 6,221,839). In a related experiment, the PC-3 conditioned media can be pre-treated with a neuropilin composition or control Fc coupled to sepharose. The cells can be lysed, immunoprecipitated using anti-VEGFR-3 antiserum, and analyzed by Western blot using anti-phosphotyrosine antibodies as previously described. The percent inhibition of VEGF-C binding and downstream VEGFR-3 autophosphorylation as a result of neuropilin sequestering of VEGF-C can be determined in this more biologically relevant situation.

The above experiments will also be carried out with relevant semaphorin proteins in conjunction with the neuropilin composition of the invention to determine the effects of another natural ligand for the neuropilin receptor on blocking VEGF-C/neuropilin receptor interactions. If the VEGF-C and semaphorin bind neuropilins in the same site on the receptor, there will be a subsequent increase in VEGF-C binding to VEGFR-3 and VEGFR-3 phosphorylation, due to the increase in VEGF-C unbound to the neuropilin-Fc. However, if the semaphorins and VEGF-C bind at different sites on the neuropilin receptor and do not inhibit each other's binding, then the amount of VEGF-C binding to VEGFR-3 will be comparable to binding in the absence of the semaphorins, i.e. with neuropilin-Fc alone. This assay will further define VEGF-C/neuropilin interactions.

The aforementioned in vitro cell-free and cell-based assays can also be performed with putative modulator compounds, e.g. cytokines that affect VEGF-C secretion (TNFa, TGFb, PDGF, TGFa, FGF-4, EGF, IL-1a IL-1b, IL-6) to determine the efficacy of the neuropilin composition at blocking VEGF-C activity in the presence of VEGF-C modulators which are biologically active in situations of inflammation and tumor growth, comparing the neuropilin composition to current experimental cancer therapeutics.

### EXAMPLE 4

### Effects of Neuropilin-2/VEGF-C Binding on VEGF-C Related Biological Functions

VEGF-C is intimately involved with many functions of lymphangiogenesis and endothelial cell growth. The influence of NRP-2 on such VEGF-C functions in vivo is investigated using the following assays:

A. Cell Migration Assay

For example, human microvascular endothelial cells (HMVEC) express VEGFR-3 and NRP-2, and such cells can be used to investigate the effect of soluble and membrane bound neuropilin receptors on such cells. Since neuropilins and VEGF/VEGFR interactions are thought to play a role in migration of cells, a cell migration assay using HMVEC or other suitable cells can be used to demonstrate stimulatory or inhibitory effects of neuropilin molecules.

Using a modified Boyden chamber assay, polycarbonate filter wells (Transwell, Costar, 8 micrometer pore) are coated with 50 micrograms/ml fibronectin (Sigma), 0.1% gelatin in PBS for 30 minutes at room temperature, followed by equilibration into DMEM/0.1% BSA at 37 degrees C. for 1 hour. HMVEC (passage 4-9, 1×10⁵ cells) naturally expressing VEGFR-3 and neuropilin receptors or endothelial cell lines recombinantly expressing VEGFR-3 and/or NRP-2 are plated in the upper chamber of the filter well and allowed to migrate to the undersides of the filters, toward the bottom chamber of the well, which contains serum-free media supplemented with prepro-VEGF-C, or enzymatically processed VEGF-C, in the presence of varying concentrations of neuropilin-1-Fc, neuropilin-2-Fc, and VEGFR-3-Fc protein. After 5 hours, cells adhering to the top of the transwell are removed with a cotton swab, and the cells that migrate to the underside of the filter are fixed and stained. For quantification of cell numbers, 6 randomly selected 400× microscope fields are counted per filter.

In another variation, the migration assay described above is carried out using porcine aortic endothelial cells (PAEC) stably transfected with constructs such as those described previously, to express NRP-2, VEGFR-3, or both NRP-2 and VEGFR-3 (i.e. PAE/NRP-2, PAE/VEGFR-3, or PAE/NRP-2/VEGFR-3). PAEC are transfected using the method described in Soker et al. (Cell 92:735-745. 1998). Transfected PAEC (1.5×10⁴ cells in serum free F12 media supplemented with 0.1% BSA) are plated in the upper wells of a Boyden chamber prepared with fibronectin as described above. Increasing concentrations of VEGF-C or VEGF-D are added to the wells of the lower chamber to induce migration of the endothelial cells. After 4 hrs, the number of cells migrating through the filter is quantitated by phase microscopy.

An increase in migration and chemotaxis of NRP-2/VEGFR-3 double transfectants over NRP-2 or VEGFR-3 single transfectants indicates that the presence of neuropilin-2 enhances the ability of VEGF-C or VEGF-D to signal through VEGFR-3 and stimulate downstream biological effects, particularly cell migration and, likely, angiogenesis or lymphangiogenesis.

Additionally, the porcine aortic endothelial cell migration assay is used to identify modulators of NRP-2/NEGFR-3/VEGF-C mediated stimulation of endothelial cells. Migration of PAE/NRP-2/VEGFR-3 expressing cells is assessed after the addition of compositions, such as soluble receptor peptides, proteins or other small molecules (e.g. monoclonal and bispecific antibodies or chemical compounds), to the lower wells of the Boyden chamber in combination with VEGF-C ligand. A decrease in migration as a result of the addition of any of the peptides, proteins or small molecules identifies that composition as an inhibitor of NRP-2/VEGFR-3 mediated chemotaxis.

B. Mitogen Assay

Embyronic endothelial cells expressing VEGFR-3 alone, NRP-2 alone, or both VEGFR-3 and NRP-2 are cultured in the presence or absence of VEGF-C polypeptides, and potential modulators of this interactions such as semaphorins, more particularly Sema3F, as well as cytokines which may include but are not limited to TGF-β, TNF-α, IL-1α and IL-1β, IL-6, and PDGF, known to upregulate VEGF-C activity, to assay effects on cell growth using any cell growth or migration assay, such as assays that measure increase in cell number or assays that measure tritiated thymidine incorporation. See, e.g., Thompson et al., Am. J. Physiol. Heart Circ. Physiol., 281: H396-403 (2001).

### EXAMPLE 5

### Angiogenesis Assays

There continues to be a long-felt need for additional agents that can stimulate angiogenesis, e.g., to promote wound healing, or to promote successful tissue grafting and transplantation, as well as agents to inhibit angiogenesis (e.g., to inhibit growth of tumors). Moreover, various angiogenesis stimulators and inhibitors may work in concert through the same or different receptors, and on different portions of the circulatory system (e.g., arteries or veins or capillaries; vascular or lymphatic). Angiogenesis assays are employed to measure the effects of neuropilin/VEGF-C interactions, on angiogenic processes, alone or in combination with other angiogenic and anti-angiogenic factors to determine preferred combination therapy involving neuropilins and other modulators. Exemplary procedures include the following.

A. In vitro Assays for Angiogenesis

1. Sprouting Assay

HMVEC cells (passage 5-9) are grown to confluency on collagen coated beads (Pharmacia) for 5-7 days. The beads are plated in a gel matrix containing 5.5 mg/ml fibronectin (Sigma), 2 units/ml thrombin (Sigma), DMEM/2% fetal bovine serum (FBS) and the following test and control proteins: 20 ng/ml VEGF, 20 ng/ml VEGF-C, or growth factors plus 10 micrograms/ml neuropilin-2-Fc, and several combinations of angiogenic factors and Fc fusion proteins. Serum free media supplemented with test and control proteins is added to the gel matrix every 2 days and the number of endothelial cell sprouts exceeding bead length are counted and evaluated.

2. Migration Assay

The transwell migration assay previously described may also be used in conjunction with the sprouting assay to determine the effects the neuropilin compositions of the invention have on the interactions of VEGF-C activators and cellular function. The effects of VEGF-Cs on cellular migration are assayed in response the neuropilin compositions of the invention, or in combination with known angiogenic or anti-angiogenic agents. A decrease in cellular migration due to the presence of the neuropilins after VEGF-C stimulation indicates that the invention provides a method for inhibiting angiogeneis.

This assay may also be carried out with cells that naturally express either VEGFR-3 or VEGFR-2, e.g. bovine endothelial cells which preferentially express VEGFR-2. Use of naturally occurring or transiently expressing cells displaying a specific receptor may determine that the neuropilin composition of the invention may be used to preferentially treat diseases involving aberrant activity of either VEGFR-3 or VEGFR-2.

B. In vivo Assays for Angiogenesis

1. Chorioallantoic Membrane (CAM) Assay

Three-day old fertilized white Leghorn eggs are cracked, and chicken embryos with intact yolks are carefully placed in 20×100 mm plastic Petri dishes. After six days of incubation in 3% CO₂ at 37 degrees C., a disk of methylcellulose containing VEGF-C and various combinations of the neuropilin compositions, VEGFR-3, and neuropilin-2 and VEGFR-3 complexes, dried on a nylon mesh (3×3 mm) is implanted on the CAM of individual embryos, to determine the influence of neuropilins on vascular development and potential uses thereof to promote or inhibit vascular formation. The nylon mesh disks are made by desiccation of 10 microliters of 0.45% methylcellulose (in H₂O). After 4-5 days of incubation, embryos and CAMs are examined for the formation of new blood vessels and lymphatic vessels in the field of the implanted disks by a stereoscope. Disks of methylcellulose containing PBS are used as negative controls. Antibodies that recognize both blood and lymphatic vessel cell surface molecules are used to further characterize the vessels.

2. Corneal Assay

Corneal micropockets are created with a modified von Graefe cataract knife in both eyes of male 5- to 6-week-old C57BL6/J mice. A micropellet (0.35×0.35 mm) of sucrose aluminum sulfate (Bukh Meditec, Copenhagen, Denmark) coated with hydron polymer type NCC (IFN Science, New Brunswick, N.J.) containing various concentrations of VEGF molecules (especially VEGF-C or VEGF-D) alone or in combination with: i) factors known to modulate vessel growth (e.g., 160 ng of VEGF, or 80 ng of FGF-2); ii) neuropilin polypeptides outlined above; or iii) neuropilin polypeptides in conjunction with natural neuropilin ligands such as semaphorins, e.g. . Sema-3C and Sema3F, is implanted into each pocket. The pellet is positioned 0.6-0.8 mm from the limbus. After implantation, erythromycin/ophthamic ointment is applied to the eyes. Eyes are examined by a slit-lamp biomicroscope over a course of 3-12 days. Vessel length and clock-hours of circumferential neovascularization and lymphangiogenesis are measured. Furthermore, eyes are cut into sections and are immunostained for blood vessel and/or lymphatic markers (LYVE-1 [Prevo et al., J. Biol. Chem., 276: 19420-19430 (2001)], podoplanin [Breiteneder-Geleff et al., Am. J. Pathol., 154: 385-94 (1999).] and VEGFR-3) to further characterize affected vessels.

### EXAMPLE 6

### In vivo Tumor Models

There is mounting evidence that neuropilin receptors may play a significant role in tumor progression. Neuropilin-1 receptors are found in several tumor cell lines and transfection of NRP-1 into AT2.1 cells can promote tumor growth and vascularization (Miao et al, FASEB J. 14: 2532-39. 2000). Additionally, investigation of neuropilin-2 expression in carcinoid tumors, slowly developing tumors derived from neuroendocrine cells in the digestive tract, illustrates that neuropilin-2 is actually expressed in normal tissue surrounding the tumor, but not in the center of the tumor itself (Cohen et al, Biochem. Biophys. Res. Comm. 284: 395-403. 2001), and it is established that neuroendocrine cells secrete VEGF-C, VEGF-D, and express VEGFR-3 on their cell surface (Partanen, et al., FASEB J 14:2087-96. 2000). Differential expression levels of these neuropilins in association with VEGF molecules, which are often correlative with vascular density and tumor progression, in and around tumors could be indicative of tumor progression or regression.

A. Ectopic Tumor Implantation

Six- to 8-week-old nude (nu/nu) mice (SLC, Shizuoka, Japan) undergo subcutaneous transplantation of C6 rat glioblastoma cells or PC-3 prostate cancer cells in 0.1 mL phosphate-buffered saline (PBS) on the right flank. The neuropilin polypeptides outlined previously are administered to the animals at various concentrations and dosing regimens. Tumor size is measured in 2 dimensions, and tumor volume is calculated using the formula, width2×length/2. After 14 days, the mice are humanely killed and autopsied to evaluate the quantity and physiology of tumor vasculature in response to VEGF-C inhibition by neuropilin polypeptides.

It will be apparent that the assay can also be performed using other tumor cell lines implanted in nude mice or other mouse strains. Use of wild type mice implanted with LLC lung cancer cells and B16 melanoma cells is specifically contemplated.

B Orthotopic Tumor Implantation

Approximately 1×10⁷ MCF-7 breast cancer cells in PBS are inoculated into the fat pads of the second (axillar) mammary gland of ovarectomized SCID mice or nude mice, carrying s.c. 60-day slow-release pellets containing 0.72 mg of 17β-estradiol (Innovative Research of America). The ovarectomy and implantation of the pellets are done 4-8 days before tumor cell inoculation. The neuropilin polypeptides and VEGF-C polypeptides outlined previously, as well as semaphorins, specifically Sema3C and Sema3F, are administered to the animals at various concentrations and dosing regimens. Tumor size is measured in 2 dimensions, and tumor volume is calculated using the formula, width 2×length/2. After 14 days, the mice are humanely killed and autopsied to evaluate the quantity and physiology of tumor vasculature.

A similar protocol is employed wherein PC-3 cells are implanted into the prostate of male mice.

C. Lymphatic Metastasis Model

VEGF-C/VEGFR3 interactions are often associated in adult tissue with the organization and growth of lymphatic vessels, thus the presence of neuropilin receptor at these sites may be involved in the metastatic nature of some cancers. The following protocol indicates the ability of neuropilin polypeptides, especially neuropilin-2 polypeptides, or fragments thereof for inhibition of lymphatic metastasis.

MDA-MB-435 breast cancer cells are injected bilaterally into the second mammary fat pads of athymic, female, eight week old nude mice. The cells often metastasize to lymph node by 12 weeks. Initially, the role of neuropilin-2 binding to VEGF-C and VEGFR-3 in tumor metastasis can be assessed using modulators of neuropilin-VEGF-C binding determined previously, especially contemplated are the semaphorins. A decrease in metastasis correlating with NRP-2 blockade indicates NRP-2 is critical in tumor metastasis. The modulators of neuropilin-VEGF-C binding determined previously [by the invention] are then administered to the animals at various concentrations and dosing regimens. Moreover, the neuropilin-2 polypeptides are administered in combination with other materials for reducing tumor metastasis. See, e.g., International Patent Publication No. WO 00/21560, incorporated herein by reference in its entirety. Mice are sacrificed after 12 weeks and lymph nodes are investigated by histologic analysis. Decrease in lymphatic vessels and tumor spread as a result of administration of the neuropilin compositions indicate the invention may be a therapeutic compound in the prevention of tumor metastasis.

### EXAMPLE 7

### Assessment of VEGF-C on Growth Cone Collapse by Collagen Repulsion Assay

The constitutive expression of semaphorins in the central nervous system has been proposed as a primary factor in the lack of regeneration of nerves in this area. Regeneration of peripheral nerves after nerve insult, such as sciatic nerve crush, is made possible by the downregulation of semaphorin-3A expression immediately following injury. Sema3A expression returns to baseline levels after approximately 36 days following injury, but this extended period of decreased semaphorin expression allows for the growth and regeneration of the peripheral nerve into the area of damage before the regrowth is halted by semaphorin activity (reviewed in Pasterkamp and Verhaagen, Brain Res. Rev. 35: 36-54. 2000). While numerous semaphorins are extensively expressed in the CNS and PNS, semaphorin-3F, the primary ligand for neuropilin-2, demonstrates wide distribution in human brain, and has even been found to be overexpressed in certain areas of the brain in Alzheimer's patients (Hirsch et al, Brain Res. 823:67-79. 1999). The newly discovered interaction of VEGF-C binding to NRP-2 may provide a factor for specifically inhibiting the actions of sema-3F activity in halting neural regeneration in many neurodegenerative diseases such as Alzheimer's or macular degeneration.

Superior cervical ganglia (SCG) are dissected out of E13.5 or E15.5-17.5 rat or mouse embryos according to the method of Chen et al (Neuron, 25:43-56. 2000) and Giger et al (Neuron, 25:29-41. 2000) for use in a collagen repulsion assay. Following dissection, hindbrain-midbrain junction explants are co-cultured with COS cells recombinantly modified to express Alkaline phosphatase conjugated Sema3F or mock transfected COS cells in collagen matrices in culture medium [OPTI-MEM and F12 at 70:25, supplemented with 1% P/S, Glutamax (Gibco), 5% FCS and 40 mM glucose] for 48 h. Neurite extension is quantitated using the protocol outlined by Giger et al (Neuron, 25:29-41. 2000), briefly described by determining the percentage of neurite extension beyond a defined point in the culture matrix. Neurite extension can be measured in the presence of varying concentrations of a VEGF-C composition as compared to in the absence of a VEGF-C composition and the subsequent increase of neurite extension as a result of VEGF-C addition to the culture and blockade of Sema3F interaction with neuropilin-2 can be assessed.

The effects of Sema3F inhibition as a result of the present invention may be extrapolated into treatments for several diseases wherein neuronal regeneration is prohibited by the presence of semaphorins, for example scarring after cranial nerve damage, and perhaps in the brains of Alzheimer's patients.

Variations to the examples given above will be apparent and are considered aspects of the invention within the claims.

For example, the materials and methods described in the preceding Examples are useful and readily adapted for screening for new modulators of the polypeptide interactions described herein, and for demonstrating the effects of such new modulators in cell-based systems and in vivo. In other words, the procedures in the materials and methods of the Examples are useful for identifying modulators and screening the modulators for activity in vitro and in vivo.

By way of illustration, Example 1 describes an experimental protocol wherein VEGF-C binding to neuropilins was investigated. Similar binding experiments can be performed in which a test agent is added to the binding experiment at one or more test agent concentrations, to determine if the test agent modulates (increases or decreases) the measurable binding between VEGF-C and the neuropilin. Example 2 describes an experimental protocol wherein VEGFR-3 binding to neuropilins was investigated. Similar binding experiments can be performed in which a test agent is included in the reaction to determine if the test agent modulates (increases or decreases) the measurable binding between VEGFR-3 and the neuropilin. Test agents that are identified as modulators in initial binding assays can be included in cell-based and in vivo assays that are provided in subsequent Examples, to measure the biological effects of the test agents on cells that express receptors of interest (e.g., VEGFR-3 or neuropilin-expressing cells) or on biological systems and organisms.

Similarly, a number of the Examples describe using a soluble form of neuropilin receptor or other protein in experiments that further prove binding relationships between molecules described herein for the first time. These experiments also demonstrate that molecules that bind one or both members of a ligand/receptor pair or receptor/co-receptor pair can be added to a system to modulate (especially inhibit) the ability of the binding pair to interact. For example, soluble NRP molecules are used in Example 3 to modulate (inhibit) VEGF-C or VEGF-D binding to VEGFR-3 or VEGFR-2. The disruption of VEGF-C or VEGF-D binding to their respective VEGFR receptors has practical applications for treatment of numerous diseases characterized by undesirable ligand-mediated stimulation of VEGFR-3 or VEGFR-2. Similar binding experiments can be performed in which a test agent suspected of modulating the same binding reactions is substituted for the soluble NRP molecule. In this way, the materials and methods of the Examples are used to identify and verify the therapeutic value of test agents.

Practicing the Examples using small organic or inorganic molecules, peptide libraries, and chemical compound libraries in place of the neuropilin or VEGF-C polypeptides is particularly contemplated. Small molecules and chemical compounds identified by the invention as modulators of neuropilin-VEGF-C and/or neuropilin/VEGFR-3 interactions will be useful as therapeutic compositions to treat situations of aberrant neuropilin-VEGF-C interactions, and in the manufacture of a medicament for the treatment of diseases characterized by aberrant growth, migration, or proliferation of cells mediated by VEGF-C binding to NRP-2/VEGFR-3 complexes.

The foregoing describes and exemplifies the invention but is not intended to limit the invention defined by the claims which follow.

What is claimed is:

1. A method of screening for modulators of binding between a neuropilin growth factor receptor and a VEGF-C polypeptide comprising steps of:
a) contacting a neuropilin composition that comprises a neuropilin polypeptide with a VEGF-C composition that comprises a VEGF-C polypeptide, in the presence and in the absence of a putative modulator compound;
b) detecting binding between the neuropilin polypeptide and the VEGF-C polypeptide in the presence and absence of the putative modulator compound; and
c) identifying a modulator compound based on a decrease or increase in binding between the neuropilin polypeptide and the VEGF-C polypeptide in the presence of the putative modulator compound, as compared to binding in the absence of the putative modulator compound.

2. A method according to claim 1, further comprising a step of:
(d) making a modulator composition by formulating a modulator identified according to step (c) in a pharmaceutically acceptable carrier.

3. A method according to claim 2, further comprising a step of:
(e) administering the modulator composition to an animal that comprises cells that express the neuropilin receptor, and determining physiological effects of the modulator composition in the animal.

4. A method according to claim 1 wherein the neuropilin receptor composition comprises a member selected from the group consisting of:
(a) a purified polypeptide comprising a neuropilin receptor extracellular domain that binds VEGF-C;
(b) a phospholipid membrane containing neuropilin polypeptides; and
(c) a cell recombinantly modified to express increased levels of neuropilin receptor polypeptide on the cell surface.

5. A method according to claim 1, wherein the neuropilin receptor composition comprises a polypeptide comprising a neuropilin receptor extracellular domain fragment bound to a solid support.

6. A method according to claim 1, wherein the neuropilin receptor composition comprises a polypeptide comprising a neuropilin receptor extracellular domain fragment fused to an immunoglobulin Fc fragment.

7. A method according to any one of claims 1-6, wherein the neuropilin composition comprises a mammalian neuropilin-2 polypeptide.

8. A method according to claim 7, wherein the neuropilin-2 polypeptide is human.

9. A method according to any one of claims 1-6, wherein the neuropilin composition comprises a mammalian neuropilin-1 polypeptide.

10. A method according to claim 1 wherein the VEGF-C composition comprises a purified mammalian prepro-VEGF-C polypeptide or a fragment of the prepro-VEGF-C polypeptide, that binds the neuropilin receptor.

11. A method according to claim 10, wherein the prepro-VEGF-C polypeptide is human.

12. A method according to claim 10, wherein the VEGF-C composition comprises a fragment of human prepro-VEGF-C that contains amino acids 103-227 of SEQ ID NO: 24.

13. A method according to any one of claims 10-12, wherein the VEGF-C composition comprises amino acids 32 to 227 of the human prepro-VEGF-C sequence of SEQ. ID. NO: 24.

14. A method according to claim 1, wherein the VEGF-C composition comprises a conditioned media from a cell recombinantly modified to express and secrete a VEGF-C polypeptide.

15. A method according to any one of claims 1-3, wherein the neuropilin composition comprises a cell recombinantly modified to express increased amounts of a neuropilin receptor on its surface, and wherein the detecting step comprises measuring a VEGF-C binding-induced physiological change in the cell.

16. A method for screening for selectivity of a modulator of VEGF-C biological activity, comprising steps of:
a) contacting a VEGF-C composition with a neuropilin composition in the presence and in the absence of a compound and detecting binding between the VEGF-C and the neuropilin in the presence and absence of the compound, wherein differential binding in the presence and absence of the compound identifies the compound as a modulator of binding between the VEGF-C and the neuropilin;
b) contacting a VEGF-C composition with a composition comprising a VEGF-C binding partner in the presence and in the absence of the compound and detecting binding between the VEGF-C and the binding partner in the presence and absence of the compound, wherein differential binding in the presence and absence of the compound identifies the compound as a modulator of binding between the VEGF-C and the binding partner; and wherein the binding partner is selected from the group consisting of:
(i) a polypeptide comprising a VEGFR-3 extracellular domain; and
(ii) a polypeptide comprising a VEGFR-2 extracellular domain; and
(c) identifying the selectivity of the modulator compound in view of the binding detected in steps (a) and (b).

17. A method for screening for selectivity of a modulator of neuropilin biological activity, comprising steps of:
a) contacting a neuropilin composition with a VEGF-C composition in the presence and in the absence of a compound and detecting binding between the neuropilin and the VEGF-C in the presence and absence of the compound, wherein differential binding in the presence and absence of the compound identifies the compound as a modulator of binding between the neuropilin and the VEGF-C;
b) contacting a neuropilin composition with a composition comprising a neuropilin binding partner in the presence and in the absence of the compound and detecting binding between the neuropilin and the binding partner in the presence and absence of the compound, wherein differential binding in the presence and absence of the compound identifies the compound as a modulator of binding between the neuropilin and the binding partner; and wherein the binding partner is a polypeptide comprising an amino acid sequence selected from the group consisting of:
an amino acid sequence of a semaphorin 3 polypeptide; a VEGF-A amino acid sequence, a VEGF-B amino acid sequence, a VEGF-D amino acid sequence, a PlGF-2 amino acid sequence, a VEGFR-1 amino acid sequence, a VEGFR-2 amino acid sequence, a VEGFR-3 amino acid sequence; and an amino acid sequence of a plexin polypeptide; and
c) identifying the selectivity of the modulator compound in view of the binding detected in steps (a) and (b).

18. A method according to claim 17 wherein the binding partner is a human semaphorin.

19. A method of screening for modulators of binding between a neuropilin growth factor receptor and a VEGFR-3 polypeptide comprising steps of:
a) contacting a neuropilin composition with a VEGFR-3 composition in the presence and in the absence of a putative modulator compound;
b) detecting binding between the neuropilin and the VEGFR-3 in the presence and absence of the putative modulator compound; and
c) identifying a modulator compound based on a decrease or increase in binding between the neuropilin composition and the VEGFR-3 composition in the presence of the putative modulator compound, as compared to binding in the absence of the putative modulator compound.

20. A method according to claim 19 wherein the VEGFR-3 composition comprises a member selected from the group consisting of:
(a) a purified polypeptide comprising a VEGFR-3 receptor extracellular domain that binds VEGF-C;
(b) a phospholipid membrane containing VEGFR-3 polypeptides; and
(c) a cell recombinantly modified to express increased levels of VEGFR-3 receptor on the cell surface.

21. A method according to claim 19, wherein the VEGFR-3 composition comprises a VEGFR-3 extracellular domain fragment bound to a solid support.

22. A method according to claim 19, wherein the VEGFR-3 composition comprises a VEGFR-3 extracellular domain fragment fused to an immunoglobulin Fc fragment.

23. A method according to any one of claims 19-22, wherein the VEGFR-3 is a mammalian VEGFR-3.

24. A method according to claim 23, wherein the VEGFR-3 is human.

25. A method for screening for selectivity of a modulator of VEGFR-3 biological activity, comprising steps of:
a) contacting a VEGFR-3 composition with a neuropilin composition in the presence and in the absence of a compound and detecting binding between the VEGFR-3 and the neuropilin in the presence and absence of the compound, wherein differential binding in the presence and absence of the compound identifies the compound as a modulator of binding between the VEGFR-3 and the neuropilin;
b) contacting a VEGFR-3 composition with a composition comprising a VEGFR-3 binding partner in the presence and in the absence of the compound and detecting binding between the VEGFR-3 and the binding partner in the presence and absence of the compound, wherein differential binding in the presence and absence of the compound identifies the compound as a modulator of binding between the VEGFR-3 and the binding partner; and wherein the binding partner is selected from the group consisting of:
(i) a polypeptide comprising a VEGF-C polypeptide; and
(ii) a polypeptide comprising a VEGF-D polypeptide; and
c) identifying the selectivity of the modulator compound in view of the binding detected in steps (a) and (b).

26. A method of modulating growth, migration, or proliferation of cells in a mammalian organism, comprising a step of:
(a) identifying a mammalian organism having cells that express a neuropilin receptor; and
(b) administering to said mammalian organism a composition, said composition comprising a neuropilin polypeptide or fragment thereof that binds to the VEGF-C polypeptide;
wherein the composition is administered in an amount effective to modulate growth, migration, or proliferation of cells that express neuropilin in the mammalian organism.

27. A method according to claim 26, wherein the mammalian organism is human.

28. A method according to claim 23, further comprising administering a second agent to the patient for modulating endothelial growth, migration, or proliferation through a neuropilin receptor, said second agent comprising a polypeptide comprising an amino acid sequence selected from the group consisting of: a VEGF-A amino acid sequence, a VEGF-B amino acid sequence, a VEGF-D amino acid sequence, a VEGF-E amino acid sequence, a PlGF amino acid sequence, a semaphorin 3A amino acid sequence, semaphorin 3C amino acid sequence, and a semaphorin 3F amino acid sequence.

29. A method of modulating growth, migration, or proliferation of cells in a mammalian organism, comprising steps of:
(a) identifying a mammalian organism having cells that express a neuropilin receptor; and
(b) administering to said mammalian organism a composition, said composition comprising a bispecific antibody specific for the neuropilin receptor and for a VEGF-C polypeptide,
wherein the composition is administered in an amount effective to modulate growth, migration, or proliferation of cells that express the neuropilin receptor in the mammalian organism.

30. A bispecific antibody which specifically binds to a neuropilin receptor and a VEGF-C polypeptide.

31. A method of modulating growth, migration, or proliferation of cells in a mammalian organism, comprising steps of:
(a) identifying a mammalian organism having cells that express a neuropilin receptor and a VEGFR-3 polypeptide; and
(b) administering to said mammalian organism a composition, said composition comprising a bispecific antibody specific for the neuropilin receptor and a VEGFR-3 polypeptide,
wherein the composition is administered in an amount effective to modulate growth, migration, or proliferation of cells that express the neuropilin receptor and the VEGFR-3 polypeptide in the mammalian organism.

32. A bispecific antibody which specifically binds to a neuropilin receptor and a VEGFR-3 polypeptide.

33. A method of modulating neuronal growth, or neuronal scarring in a mammalian organism, comprising a step of:
(a) identifying a mammalian organism having neuronal cells that express a neuropilin receptor; and
(b) administering to said mammalian organism a composition, said composition comprising a VEGF-C polypeptide or fragment thereof that binds to the neuropilin receptor,
wherein said polypeptide or fragment is administered in an amount effective to modulate the growth, survival or migration of the neuronal cells.

34. A method according to claim 33, wherein the mammalian organism is human.

35. A method according to claim 33, wherein the neuronal cells comprise neuronal cells that express neuropilin-2.

36. A method according to any one of claims 33, wherein the organism has a disease or condition characterized by aberrant growth of neuronal cells, neuronal scarring, or neural degeneration.

37. A method according to claim 36, wherein the disease comprises a neurodegenerative disorder.

38. A method according to claim 37, wherein the disease is Alzheimer's disease.

39. A polypeptide comprising a fragment of a VEGF-C that binds to a neuropilin receptor, for use in the manufacture of a medicament for the treatment of diseases characterized by aberrant growth, migration, or proliferation of cells that express a neuropilin receptor.


SEQUENCE LISTING (33 sequences)

SEQ ID NO: 1 - DNA, 2772 bp, Homo sapiens
  CDS (1)..(2772)
atggagagggggctgccgctcctctgcgccgtgctcgccctcgtcctcgccccggccggcgcttttcgcaacgatgaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggagacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaacccatcctcatcaccatcatagccatgagcgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 2 - protein, 923 aa, Homo sapiens
MERGLPLLCAVLALVLAPAGAFRNDECGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLEPILITIIAMSALGVLLGAVCGVVLYCACWHNGMSERNLSALENYNFELVDGVKLKKDKLNTQSTYSEA

SEQ ID NO: 3 - DNA, 2781 bp, Homo sapiens
  CDS (1)..(2781)
atggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactacgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggtcagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtaaaaacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatga

SEQ ID NO: 4 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVKTLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 5 - DNA, 3652 bp, Mus musculus
  CDS (348)..(3119)
  misc_feature (348)..(410): Signal Peptide
tttttttttttttttttttttttttttttttttttcctccttcttcttcttcctgagacatggcccgggcagtggctcctggaagaggaacaagtgtgggaaaagggagaggaaatcggagctaaatgacaggatgcaggcgacttgagacacaaaaagagaagcgcttctcgcgaattcaggcattgcctcgccgctagccttccccgccaagacccgctgaggattttatggttcttaggcggacttaagagcgtttcggattgttaagattatcgtttgctggtttttcgtccgcgcaatcgtgttctcctgcggctgcctggggactggcttggcgaaggaggatggagagggggctgccgttgctgtgcgccacgctcgcccttgccctcgccctggcgggcgctttccgcagcgacaaatgtggcgggaccataaaaatcgaaaacccagggtacctcacatctcccggttaccctcattcttaccatccaagtgagaagtgtgaatggctaatccaagctccggaaccctaccagagaatcataatcaacttcaacccacatttcgatttggaggacagagactgcaagtatgactacgtggaagtaattgatggggagaatgaaggcggccgcctgtgggggaagttctgtgggaagattgcaccttctcctgtggtgtcttcagggccctttctcttcatcaaatttgtctctgactatgagacacatggggcagggttttccatccgctatgaaatcttcaagagagggcccgaatgttctcagaactatacagcacctactggagtgataaagtcccctgggttccctgaaaaataccccaactgcttggagtgcacctacatcatctttgcaccaaagatgtctgagataatcctggagtttgaaagttttgacctggagcaagactcgaatcctcccggaggaatgttctgtcgctatgaccggctggagatctgggatggattccctgaagttggccctcacattgggcgttattgtgggcagaaaactcctggccggatccgctcctcttcaggcgttctatccatggtcttttacactgacagcgcaatagcaaaagaaggtttctcagccaactacagtgtgctacagagcagcatctctgaagattttaagtgtatggaggctctgggcatggaatctggagagatccattctgatcagatcactgcatcttcacagtatggtaccaactggtctgtagagcgctcccgcctgaactaccctgaaaatgggtggactccaggagaagactcctacaaggagtggatccaggtggacttgggcctcctgcgattcgttactgctgtagggacacagggtgccatttccaaggaaaccaagaagaaatattatgtcaagacttacagagtagacatcagctccaacggagaggactggatctccctgaaagagggaaataaagccattatctttcagggaaacaccaaccccacagatgttgtcttaggagttttctccaaaccactgataactcgatttgtccgaatcaaacctgtatcctgggaaactggtatatctatgagatttgaagtttatggctgcaagataacagattatccttgctctggaatgttgggcatggtgtctggacttatttcagactcccagattacagcatccaatcaagccgacaggaattggatgccagaaaacatccgtctggtgaccagtcgtaccggctgggcactgccaccctcaccccacccatacaccaatgaatggctccaagtggacctgggagatgagaagatagtaagaggtgtcatcattcagggtgggaagcaccgagaaaacaaggtgttcatgaggaagttcaagatcgcctatagtaacaatggctctgactggaaaactatcatggatgacagcaagcgcaaggctaagtcgttcgaaggcaacaacaactatgacacacctgagcttcggacgttttcacctctctccacaaggttcatcaggatctaccctgagagagccacacacagtgggcttgggctgaggatggagctactgggctgtgaagtggaagcacctacagctggaccaaccacacccaatgggaacccagtgcatgagtgtgacgacgaccaggccaactgccacagtggcacaggtgatgacttccagctcacaggaggcaccactgtcctggccacagagaagccaaccattatagacagcaccatccaatcagagttcccgacatacggttttaactgcgagtttggctggggctctcacaagacattctgccactgggagcatgacagccatgcacagctcaggtggagtgtgctgaccagcaagacagggccgattcaggaccatacaggagatggcaacttcatctattcccaagctgatgaaaatcagaaaggcaaagtagcccgcctggtgagccctgtggtctattcccagagctctgcccactgtatgaccttctggtatcacatgtccggctctcatgtgggtacactgagggtcaaactacgctaccagaagccagaggaatatgatcaactggtctggatggtggttgggcaccaaggagaccactggaaagaaggacgtgtcttgctgcacaaatctctgaaactatatcaggttatttttgaaggtgaaatcggaaaaggaaaccttggtggaattgctgtggatgatatcagtattaacaaccatatttctcaggaagactgtgcaaaaccaacagacctagataaaaagaacacagaaattaaaattgatgaaacagggagcactccaggatatgaaggagaaggggaaggtgacaagaacatctccaggaagccaggcaatgtgcttaagaccctggatcccatcctgatcaccatcatagccatgagtgccctgggagtactcctgggtgcagtctgtggagttgtgctgtactgtgcctgttggcacaatgggatgtcagaaaggaacctatctgccctggagaactataactttgaacttgtggatggtgtaaagttgaaaaaagataaactgaacccacagagtaattactcagaggcgtgaaggcacggagctggagggaacaagggaggagcacggcaggagaacaggtggaggcatggggactctgttactctgctttcactgtaagctgggaagggcggggactctgttactccgctttcactgtaagctcggaagggcatccacgatgccatgccaggcttttctcaggagcttcaatgagcgtcacctacagacacaagcaggtgactgcggtaacaacaggaatcatgtacaagcctgctttcttctcttggtttcatttgggtaatcagaagccatttgagaccaagtgtgactgacttcatggttcatcctactagcccccttttttcctctctttctccttaccctgtggtggattcttctcggaaactgcaaaatccaagatgctggcactaggcgttattcagtgggcccttttgatggacatgtgacctgtagcccagtgcccagagcatattatcataaccacatttcaggggacgccaacgtccatccacctttgcatcgctacctgcagcgagcacagg

SEQ ID NO: 6 - protein, 923 aa, Mus musculus
  misc_feature (348)..(410): Signal Peptide
MERGLPLLCATLALALALAGAFRSDKCGGTIKIENPGYLTSPGYPHSYHPSEKCEWLIQAPEPYQRIIINFNPHFDLEDRDCKYDYVEVIDGENEGGRLWGKFCGKIAPSPVVSSGPFLFIKFVSDYETHGAGFSIRYEIFKRGPECSQNYTAPTGVIKSPGFPEKYPNCLECTYIIFAPKMSEIILEFESFDLEQDSNPPGGMFCRYDRLEIWDGFPEVGPHIGRYCGQKTPGRIRSSSGVLSMVFYTDSAIAKEGFSANYSVLQSSISEDFKCMEALGMESGEIHSDQITASSQYGTNWSVERSRLNYPENGWTPGEDSYKEWIQVDLGLLRFVTAVGTQGAISKETKKKYYVKTYRVDISSNGEDWISLKEGNKAIIFQGNTNPTDVVLGVFSKPLITRFVRIKPVSWETGISMRFEVYGCKITDYPCSGMLGMVSGLISDSQITASNQADRNWMPENIRLVTSRTGWALPPSPHPYTNEWLQVDLGDEKIVRGVIIQGGKHRENKVFMRKFKIAYSNNGSDWKTIMDDSKRKAKSFEGNNNYDTPELRTFSPLSTRFIRIYPERATHSGLGLRMELLGCEVEAPTAGPTTPNGNPVHECDDDQANCHSGTGDDFQLTGGTTVLATEKPTIIDSTIQSEFPTYGFNCEFGWGSHKTFCHWEHDSHAQLRWSVLTSKTGPIQDHTGDGNFIYSQADENQKGKVARLVSPVVYSQSSAHCMTFWYHMSGSHVGTLRVKLRYQKPEEYDQLVWMVVGHQGDHWKEGRVLLHKSLKLYQVIFEGEIGKGNLGGIAVDDISINNHISQEDCAKPTDLDKKNTEIKIDETGSTPGYEGEGEGDKNISRKPGNVLKTLDPILITIIAMSALGVLLGAVCGVVLYCACWHNGMSERNLSALENYNFELVDGVKLKKDKLNPQSNYSEA

SEQ ID NO: 7 - DNA, 4769 bp, Mus musculus
  CDS (567)..(3347)
aaactggagctccaccgcggtggcggccgcccgggcaggtctagaattcagcggccgctgaattctatccagcggtcggtgcctctgcccgcgtgtgtgtcccgggtgccgggggacctgtgtcagttagcgcttctgagatcacacagctgcctaggggccgtgtgatgcccagggcaattcttggctttgatttttattattattactattattttgcgttcagctttcgggaaaccctcgtgatgttgtaggataaaggaaatgacactttgaggaactggagagaacatacacgcgtttgggtttgaagaggaaaccggtctccgcttccttagcttgctccctctttgctgatttcaagagctatctcctatgaggtggagatattccagcaagaataaaggtgaagacagactgactgccaggacccaggaggaaaacgttgatcgttagagacctttgcagaagacaccaccaggaggaaaattagagaggaaaaacacaaagacataattataggagatcccacaaacctagcccgggagagagcctctctgtcaaaaatggatatgtttcctcttacctgggttttcttagctctgtacttttcaggacacgaagtgagaagccagcaagatccaccctgcggaggtcggccgaattccaaagatgctggctacatcacttccccaggctacccccaggactatccctcccaccagaactgtgagtggattgtctacgcccccgaacccaaccagaagattgttctcaacttcaaccctcactttgaaatcgagaaacacgactgcaagtatgacttcattgagattcgggatggggacagtgagtcagctgacctcctgggcaagcactgtgggaacatcgccccgcccaccatcatctcctcaggctccgtgttatacatcaagttcacctcagactacgcccggcagggggcaggtttctctctacgctatgagatcttcaaaacaggctctgaagattgttccaagaactttacaagccccaatgggaccattgaatctccagggtttccagagaagtatccacacaatctggactgtaccttcaccatcctggccaaacccaggatggagatcatcctacagttcctgacctttgacctggagcatgaccctctacaagtgggggaaggagactgtaaatatgactggctggacatctgggatggcattccacatgttggacctctgattggcaagtactgtgggacgaaaacaccctccaaactccgctcgtccacggggatcctctccttgacctttcacacggacatggcagtggccaaggatggcttctccgcacgttactatttgatccaccaggagccacctgagaattttcagtgcaatgtccctttgggaatggagtctggccggattgctaatgaacagatcagtgcctcctccaccttctctgatgggaggtggactcctcaacagagccggctccatggtgatgacaatggctggacacccaatttggattccaacaaggagtatctccaggtggacctgcgcttcctaaccatgctcacagccattgcaacacagggagccatttccagggaaacccagaaaggctactacgtcaaatcgtacaagctggaagtcagcacaaatggtgaagattggatggtctaccggcatggcaaaaaccacaagatattccaagcgaacaatgatgcgaccgaggtggtgctaaacaagctccacatgccactgctgactcggttcatcaggatccgcccgcagacgtggcatttgggcattgcccttcgcctggagctctttggctgccgggtcacagatgcaccctgctccaacatgctggggatgctctcgggcctcattgctgatacccagatctctgcctcctccacccgagagtacctctggagccccagtgctgcccgcctggttagtagccgctctggctggtttcctcggaaccctcaagcccagccaggtgaagaatggcttcaggtagacctggggacacccaagacagtgaaaggggtcatcatccagggagcccgaggaggagacagcatcactgccgtggaagccagggcgtttgtacgcaagttcaaagtctcctacagcctaaatggcaaggactgggaatatatccaggaccccaggactcagcagacaaagctgtttgaagggaacatgcactatgacacccctgacatccgaaggttcgatcctgttccagcgcagtatgtgcgggtgtacccagagaggtggtcgccagcaggcatcgggatgaggctggaggtgctgggctgtgactggacagactcaaagcccacagtggagacgctgggacccaccgtgaagagtgaagagactaccaccccatatcccatggatgaggatgccaccgagtgtggggaaaactgcagctttgaggatgacaaagatttgcaacttccttcaggattcaactgcaactttgattttccggaagagacctgtggttgggtgtacgaccatgccaagtggctccggagcacgtggatcagcagcgctaaccccaatgacagaacatttccagatgacaagaacttcttgaaactgcagagtgatggccgacgagagggccagtacgggcggctcatcagcccaccggtgcacctgccccgaagccctgtgtgcatggagttccagtaccaagccatgggcggccacggggtggcactgcaggtggttcgggaagccagccaggaaagcaaactcctttgggtcatccgtgaggaccagggcagcgagtggaagcacgggcgcattatcctgcccagctatgacatggagtatcagatcgtgttcgagggagtgatagggaagggacgatcgggagagatttccggcgatgacattcggataagcactgatgtcccactggagaactgcatggaacccatatcagcttttgcagtggacatcccagaaacccatgggggagagggctatgaagatgagattgatgatgaatatgaaggagattggagcaactcttcttcctctacctcaggggctggtgacccctcatctggcaaagaaaagagctggctgtacaccctagatcccattctgatcaccatcatcgccatgagctcgctgggggtcctgctgggggccacctgtgcgggcctcctcctttactgcacctgctcctattcgggtctgagttcgaggagctgcaccacactggagaactacaactttgagctctacgatggcctcaagcacaaggtcaagatcaatcatcagaagtgctgctcggaggcatgaccgattgtgtctggatcgcttctggcgtttcattccagtgagaggggctagcgaagattacagttttgttttgttttgttttgttttccctttggaaactgaatgccataatctggatcaaagtgttccagaatactgaaggtatggacaggacagacaggccagtctagggagaaagggagatgcagctgtgaaggggatcgttgcccaccaggactgtggtggccaagtgaatgcaggaaccgggcccggaattccggctctcggctaaaatctcagctgcctctggaaaggctcaaccatactcagtgccaactcagactctgttgctgtggtgtcaacatggatggatcatctgtaccttgtatttttagcagaattcatgctcagatttctttgttctgaatccttgctttgtgctagacacaaagcatacatgtccttctaaaattaatatgatcactataatctcctgtgtgcagaattcagaaatagacctttgaaaccatttgcattgtgagtgcagatccatgactggggctagtgcagcaatgaaacagaattccagaaacagtgtgttctttttattatgggaaaatacagataaaaatggccactgatgaacatgaaagttagcactttcccaacacagtgtacacttgcaaccttgttttggatttctcatacaccaagactgtgaaacacaaatttcaagaatgtgttcaaatgtgtgtgtgtgtgtgtgtgtgtgtgtgtgtgtgtgtgtatgtgtgtgtgtgtgtgtgtgcttgtgtgtttctgtcagtggtatgagtgatatgtatgcatgtgtgtatgtatatgtatgtatgtatgtatgtatgtacgtacatatgtatgtatgtatgtatgtatgtatgtatgtatatgtgtgtgtgtgtttgtgtgtgtgtgtgtttgtgtgtgtgtgtgtggtaagtgtggtatgtgtgtatgcatttgtctatatgtgtatctgtgtgtctatgtgtttctgtcagtggaatgagtggcatgtgtgcatgtgtatgtatgtggatatgtgtgttgtgtttatgtgcttgtgtataagaggtaagtgtggtgtgtgtgcatgtgtctctgtgtgtgtttgtctgtgtacctctttgtataagtacctgtgtttgtatgtgggaatatgtatattgaggcattgctgtgttagtatgtttatagaaaagaagacagtctgagatgtcttcctcaatacctctccacttatatcttggatagacaaaagtaatgacaaaaaattgctggtgtgtatatggaaaagggggacacatatccatggatggtagaagtgtaaactgtgcagtcactgtggacatcaatatgcaggttcttcacaaatgtagatataaagctactatagttataccc

SEQ ID NO: 8 - protein, 926 aa, Mus musculus
MDMFPLTWVFLALYFSGHEVRSQQDPPCGGRPNSKDAGYITSPGYPQDYPSHQNCEWIVYAPEPNQKIVLNFNPHFEIEKHDCKYDFIEIRDGDSESADLLGKHCGNIAPPTIISSGSVLYIKFTSDYARQGAGFSLRYEIFKTGSEDCSKNFTSPNGTIESPGFPEKYPHNLDCTFTILAKPRMEIILQFLTFDLEHDPLQVGEGDCKYDWLDIWDGIPHVGPLIGKYCGTKTPSKLRSSTGILSLTFHTDMAVAKDGFSARYYLIHQEPPENFQCNVPLGMESGRIANEQISASSTFSDGRWTPQQSRLHGDDNGWTPNLDSNKEYLQVDLRFLTMLTAIATQGAISRETQKGYYVKSYKLEVSTNGEDWMVYRHGKNHKIFQANNDATEVVLNKLHMPLLTRFIRIRPQTWHLGIALRLELFGCRVTDAPCSNMLGMLSGLIADTQISASSTREYLWSPSAARLVSSRSGWFPRNPQAQPGEEWLQVDLGTPKTVKGVIIQGARGGDSITAVEARAFVRKFKVSYSLNGKDWEYIQDPRTQQTKLFEGNMHYDTPDIRRFDPVPAQYVRVYPERWSPAGIGMRLEVLGCDWTDSKPTVETLGPTVKSEETTTPYPMDEDATECGENCSFEDDKDLQLPSGFNCNFDFPEETCGWVYDHAKWLRSTWISSANPNDRTFPDDKNFLKLQSDGRREGQYGRLISPPVHLPRSPVCMEFQYQAMGGHGVALQVVREASQESKLLWVIREDQGSEWKHGRIILPSYDMEYQIVFEGVIGKGRSGEISGDDIRISTDVPLENCMEPISAFAVDIPETHGGEGYEDEIDDEYEGDWSNSSSSTSGAGDPSSGKEKSWLYTLDPILITIIAMSSLGVLLGATCAGLLLYCTCSYSGLSSRSCTTLENYNFELYDGLKHKVKINHQKCCSEA

SEQ ID NO: 9 - DNA, 2530 bp, Homo sapiens
  CDS (16)..(2331)
ggaattccctgcagcatgggctggttaactaggattgtctgtcttttctggggagtattacttacagcaagagcaaactatcagaatgggaagaacaatgtgccaaggctgaaattatcctacaaagaaatgttggaatccaacaatgtgatcactttcaatggcttggccaacagctccagttatcataccttccttttggatgaggaacggagtaggctgtatgttggagcaaaggatcacatattttcattcgacctggttaatatcaaggattttcaaaagattgtgtggccagtatcttacaccagaagagatgaatgcaagtgggctggaaaagacatcctgaaagaatgtgctaatttcatcaaggtacttaaggcatataatcagactcacttgtacgcctgtggaacgggggcttttcatccaatttgcacctacattgaaattggacatcatcctgaggacaatatttttaagctggagaactcacattttgaaaacggccgtgggaagagtccatatgaccctaagctgctgacagcatcccttttaatagatggagaattatactctggaactgcagctgattttatggggcgagactttgctatcttccgaactcttgggcaccaccacccaatcaggacagagcagcatgattccaggtggctcaatgatccaaagttcattagtgcccacctcatctcagagagtgacaatcctgaagatgacaaagtatactttttcttccgtgaaaatgcaatagatggagaacactctggaaaagctactcacgctagaataggtcagatatgcaagaatgactttggagggcacagaagtctggtgaataaatggacaacattcctcaaagctcgtctgatttgctcagtgccaggtccaaatggcattgacactcattttgatgaactgcaggatgtattcctaatgaactttaaagatcctaaaaatccagttgtatatggagtgtttacgacttccagtaacattttcaagggatcagccgtgtgtatgtatagcatgagtgatgtgagaagggtgttccttggtccatatgcccacagggatggacccaactatcaatgggtgccttatcaaggaagagtcccctatccacggccaggaacttgtcccagcaaaacatttggtggttttgactctacaaaggaccttcctgatgatgttataacctttgcaagaagtcatccagccatgtacaatccagtgtttcctatgaacaatcgcccaatagtgatcaaaacggatgtaaattatcaatttacacaaattgtcgtagaccgagtggatgcagaagatggacagtatgatgttatgtttatcggaacagatgttgggaccgttcttaaagtagtttcaattcctaaggagacttggtatgatttagaagaggttctgctggaagaaatgacagtttttcgggaaccgactgctatttcagcaatggagctttccactaagcagcaacaactatatattggttcaacggctggggttgcccagctccctttacaccggtgtgatatttacgggaaagcgtgtgctgagtgttgcctcgcccgagacccttactgtgcttgggatggttctgcatgttctcgctattttcccactgcaaagagacgcacaagacgacaagatataagaaatggagacccactgactcactgttcagacttacaccatgataatcaccatggccacagccctgaagagagaatcatctatggtgtagagaatagtagcacatttttggaatgcagtccgaagtcgcagagagcgctggtctattggcaattccagaggcgaaatgaagagcgaaaagaagagatcagagtggatgatcatatcatcaggacagatcaaggccttctgctacgtagtctacaacagaaggattcaggcaattacctctgccatgcggtggaacatgggttcatacaaactcttcttaaggtaaccctggaagtcattgacacagagcatttggaagaacttcttcataaagatgatgatggagatggctctaagaccaaagaaatgtccaatagcatgacacctagccagaaggtctggtacagagacttcatgcagctcatcaaccaccccaatctcaacacgatggatgagttctgtgaacaagtttggaaaagggaccgaaaacaacgtcggcaaaggccaggacataccccagggaacagtaacaaatggaagcacttacaagaaaataagaaaggtagaaacaggaggacccacgaatttgagagggcacccaggagtgtctgagctgcattacctctagaaacctcaaacaagtagaaacttgcctagacaataactggaaaaacaaatgcaatatacatgaacttttttcatggcattatgtggatgtttacaatggtgggaaattcagctgagttccaccaattataaattaaatccatgagtaactttcctaataggcttttttttcctaataccaccg

SEQ ID NO: 10 - protein, 771 aa, Homo sapiens [sequence omitted]

SEQ ID NO: 11 - DNA, 2919 bp, Homo sapiens
  CDS (236)..(2485)
tctgtgattgtggccaggcggggcaccctcggaggggagggttcggaagtggaatgcgaccccccagcctctttcccctaggggctgtaatctgatccctggggactccccccctagcctcccgccctcgccctcactgctgactcctcttccagatcctggggcagagtccagggcagctcaaggctcctccacacacacacccgctgaaccctgagcaccctgagctgctgagatggggcgggccggggctgccgccgtgatcccgggcctggccctgctctgggcagtggggctggggagtgccgcccccagccccccacgccttcggctctccttccaagagctccaggcctggcatggtctccagactttcagcctggagcgaacctgctgctaccaggccttgctggtggatgaggagcgtggacgcctgtttgtgggtgccgagaaccatgtggcctccctcaacctggacaacatcagcaagcgggccaagaagctggcctggccggcccctgtggaatggcgagaggagtgcaactgggcagggaaggacattggtactgagtgcatgaacttcgtgaagttgctgcatgcctacaaccgcacccatttgctggcctgtggcacgggagccttccacccaacctgtgcctttgtggaagtgggccaccgggcagaggagcccgtcctccggctggacccaggaaggatagaggatggcaaggggaagagtccttatgaccccaggcatcgggctgcctccgtgctggtgggggaggagctatactcaggggtggcagcagacctcatgggacgagactttaccatctttcgcagcctagggcaacgtccaagtctccgaacagagccacacgactcccgctggctcaatgagcccaagtttgtcaaggtattttggatcccggagagcgagaacccagacgacgacaaaatctacttcttctttcgtgagacggcggtagaggcggcgccggcactgggacgcctgtccgtgtcccgcgttggccagatctgccggaacgacgtgggcggccagcgcagcctggtcaacaagtggacgacgttcctgaaggcgcggctggtgtgctcggtgcccggcgtcgagggcgacacccacttcgatcagctccaggatgtgtttctgttgtcctcgcgggaccaccggaccccgctgctctatgccgtcttctccacgtccagcagcatcttccagggctctgcggtgtgcgtgtacagcatgaacgacgtgcgccgggccttcttgggaccctttgcacacaaggaggggcccatgcaccagtgggtgtcataccagggtcgcgtcccctacccgcggccaggcatgtgccccagcaagacctttggcaccttcagttccaccaaggacttcccagacgatgtcatccagtttgcgcggaaccaccccctcatgtacaactctgtcctgcccactggggggcgccctcttttcctacaagttggagccaattacaccttcactcaaattgccgcggaccgggttgcagccgctgacggacactatgacgtcctcttcattggcacagacgttggcacggtgctgaaggtgatctcggtccccaagggcagtaggcccagcgcagaggggctgctcctggaggagctgcacgtgtttgaggactcggccgctgtcaccagcatgcaaatttcttccaagaggcaccagctgtacgtagcctcgcggagcgcggtggcccagatcgcgttgcaccgctgcgctgcccacggccgcgtctgcaccgaatgctgtctggcgcgtgacccctactgcgcctgggacggggtcgcgtgcacgcgcttccagcccagtgccaagaggcggttccggcggcaagacgtaaggaatggcgaccccagcacgttgtgctccggagactcgtctcgtcccgcgctgctggaacacaaggtgttcggcgtggagggcagcagcgcctttctggagtgtgagccccgctcgctgcaggcgcgcgtggagtggactttccagcgcgcaggggtgacagcccacacccaggtgctggcagaggagcgcaccgagcgcaccgcccggggactactgctgcgcaggctgcggcgccgggactcgggcgtgtacttgtgcgccgccgtcgagcagggctttacgcaaccgctgcgtcgcctgtcgctgcacgtgttgagtgctacgcaggccgaacgactggcgcgggccgaggaggctgcgcccgccgcgccgccgggccccaaactctggtaccgggactttctgcagctggtggagccgggcggaggtggcagcgcgaactccctgcgcatgtgccgcccgcagcctgcgctgcagtcactgcccctggagtcgcggagaaagggccgtaaccggaggacccacgcccctgagcctcgcgctgagcgggggccgcgcagcgcaacgcactggtgaccagactgtccccacgccgggaaccaagcaggagacgacaggcgagagaggagccagacagaccctgaaaagaaggacgggttggggccgggcacattgggggtcaccggccgatggagacaccaaccgacaggccctggctgagggcagctgcgcgggcttatttattaacaggataacccttgaatgtagcagccccgggagggcggcacaggtcgggcgcaggattcagccggagggaagggacggggaagccgagctccagagcaacgaccagggccgaggaggtgcctggagtgcccaccctgggagacagaccccacctccttgggtagtgagcagtgagcagaaagctgtgaacaggctgggctgctggaggtggggcgaggcaggccgactgtactaaagtaacgcaataaacgcattatcagcca

SEQ ID NO: 12 - protein, 749 aa, Homo sapiens
MGRAGAAAVIPGLALLWAVGLGSAAPSPPRLRLSFQELQAWHGLQTFSLERTCCYQALLVDEERGRLFVGAENHVASLNLDNISKRAKKLAWPAPVEWREECNWAGKDIGTECMNFVKLLHAYNRTHLLACGTGAFHPTCAFVEVGHRAEEPVLRLDPGRIEDGKGKSPYDPRHRAASVLVGEELYSGVAADLMGRDFTIFRSLGQRPSLRTEPHDSRWLNEPKFVKVFWIPESENPDDDKIYFFFRETAVEAAPALGRLSVSRVGQICRNDVGGQRSLVNKWTTFLKARLVCSVPGVEGDTHFDQLQDVFLLSSRDHRTPLLYAVFSTSSSIFQGSAVCVYSMNDVRRAFLGPFAHKEGPMHQWVSYQGRVPYPRPGMCPSKTFGTFSSTKDFPDDVIQFARNHPLMYNSVLPTGGRPLFLQVGANYTFTQIAADRVAAADGHYDVLFIGTDVGTVLKVISVPKGSRPSAEGLLLEELHVFEDSAAVTSMQISSKRHQLYVASRSAVAQIALHRCAAHGRVCTECCLARDPYCAWDGVACTRFQPSAKRRFRRQDVRNGDPSTLCSGDSSRPALLEHKVFGVEGSSAFLECEPRSLQARVEWTFQRAGVTAHTQVLAEERTERTARGLLLRRLRRRDSGVYLCAAVEQGFTQPLRRLSLHVLSATQAERLARAEEAAPAAPPGPKLWYRDFLQLVEPGGGGSANSLRMCRPQPALQSLPLESRRKGRNRRTHAPEPRAERGPRSATHW

SEQ ID NO: 13 - DNA, 5177 bp, Homo sapiens
  CDS (563)..(2818)
ggactgcgaaaggagcagggttgcggagctagggctccagcctgcggccgcgcattcttgcgtctggccagccgcgagctctaagggtcggccccgcccggtccgcccccgcggctccctgccaggctctcgcgggcgcgctcggggtggggcctcgcggctggcggagatgcggccggggctgcgcggtggtgatgcgagcctgctgggcggcgcgccggggcagccggagccgcgcgccgcggcgctgtaatcggacaccaagagcgctcgcccccggcctccggccactttccattcactccgaggtgcttgattgagcgacgcggagaagagctccgggtgccgcggcactgcagcgctgagattcctttacaaagaaactcagaggaccgggaagaaagaatttcacctttgcgacgtgctagaaaataaggtcgtctgggaaaaggactggagacacaagcgcatccaaccccggtagcaaactgatgacttttccgtgctgatttctttcaacctcggtattttcccttggatattaacttgcatatctgaagaaatggcattccggacaatttgcgtgttggttggagtatttatttgttctatctgtgtgaaaggatcttcccagccccaagcaagagtttatttaacatttgatgaacttcgagaaaccaagacctctgaatacttcagcctttcccaccatcctttagactacaggattttattaatggatgaagatcaggaccggatatatgtgggaagcaaagatcacattctttccctgaatattaacaatataagtcaagaagctttgagtgttttctggccagcatctacaatcaaagttgaagaatgcaaaatggctggcaaagatcccacacacggctgtgggaactttgtccgtgtaattcagactttcaatcgcacacatttgtatgtctgtgggagtggcgctttcagtcctgtctgtacttacttgaacagagggaggagatcagaggaccaagttttcatgattgactccaagtgtgaatctggaaaaggacgctgctctttcaaccccaacgtgaacacggtgtctgttatgatcaatgaggagcttttctctggaatgtatatagatttcatggggacagatgctgctatttttcgaagtttaaccaagaggaatgcggtcagaactgatcaacataattccaaatggctaagtgaacctatgtttgtagatgcacatgtcatcccagatggtactgatccaaatgatgctaaggtgtacttcttcttcaaagaaaaactgactgacaataacaggagcacgaaacagattcattccatgattgctcgaatatgtcctaatgacactggtggactgcgtagccttgtcaacaagtggaccactttcttaaaggcgaggctggtgtgctcggtaacagatgaagacggcccagaaacacactttgatgaattagaggatgtgtttctgctggaaactgataacccgaggacaacactagtgtatggcatttttacaacatcaagctcagttttcaaaggatcagccgtgtgtgtgtatcatttatctgatatacagactgtgtttaatgggccttttgcccacaaagaagggcccaatcatcagctgatttcctatcagggcagaattccatatcctcgccctggaacttgtccaggaggagcatttacacccaatatgcgaaccaccaaggagttcccagatgatgttgtcacttttattcggaaccatcctctcatgtacaattccatctacccaatccacaaaaggcctttgattgttcgtattggcactgactacaagtacacaaagatagctgtggatcgagtgaacgctgctgatgggagataccatgtcctgtttctcggaacagatcggggtactgtgcaaaaagtggttgttcttcctactaacaactctgtcagtggcgagctcattctggaggagctggaagtctttaagaatcatgctcctataacaacaatgaaaatttcatctaaaaagcaacagttgtatgtgagttccaatgaaggggtttcccaagtatctctgcaccgctgccacatctatggtacagcctgtgctgactgctgcctggcgcgggacccttattgcgcctgggatggccattcctgttccagattctacccaactgggaaacggaggagccgaagacaagatgtgagacatggaaacccactgactcaatgcagaggatttaatctaaaagcatacagaaatgcagctgaaattgtgcagtatggagtaaaaaataacaccacttttctggagtgtgcccccaagtctccgcaggcatctatcaagtggctgttacagaaagacaaagacaggaggaaagaggttaagctgaatgaacgaataatagccacttcacagggactcctgatccgctctgttcagggttctgaccaaggactttatcactgcattgctacagaaaatagtttcaagcagaccatagccaagatcaacttcaaagttttagattcagaaatggtggctgttgtgacggacaaatggtccccgtggacctgggccagctctgtgagggctttacccttccacccgaaggacatcatgggggcattcagccactcagaaatgcagatgattaaccaatactgcaaagacactcggcagcaacatcagcagggagatgaatcacagaaaatgagaggggactatggcaagttaaaggccctcatcaatagtcggaaaagtagaaacaggaggaatcagttgccagagtcataatattttcttatgtgggtcttatgcttccattaacaaatgctctgtcttcaatgatcaaattttgagcaaagaaacttgtgctttaccaaggggaattactgaaaaaggtgattactcctgaagtgagttttacacgaactgaaatgagcatgcattttcttgtatgatagtgactagcactagacatgtcatggtcctcatggtgcatataaatatatttaacttaacccagattttatttatatctttattcaccttttcttcaaaatcgatatggtggctgcaaaactagaattgttgcatccctcaattgaatgagggccatatccctgtggtattcctttcctgctttggggctttagaattctaattgtcagtgattttgtatatgaaaacaagttccaaatccacagcttttacgtagtaaaagtcataaatgcatatgacagaatggctatcaaaagaaatagaaaaggaagacggcatttaaagttgtataaaaacacgagttattcataaagagaaaatgatgagtttttatggttccaatgaaatatcttcccctttttttaagattgtaaaaataatcagttactggtatctgtcactgacctttgtttccttattcaggaagataaaaatcagtaacctaccccatgaagatatttggtgggagttatatcagtgaagcagtttggtttatattcttatgttatcaccttccaaacaaaagcacttactttttttggaagttatttaatttattttagactcaaagaatataatcttgcactactcagttattactgtttgttctcttattccctagtctgtgtggcaaattaaacaatataagaaggaaaaatttgaagtattagacttctaaataaggggtgaaatcatcagaaagaaaaatcaaagtagaaactactaattttttaagaggaatttataacaaatatggctagttttcaacttcagtactcaaattcaatgattcttccttttattaaaaccagtctcagatatcatactgatttttaagtcaacactatatattttatgatcttttcagtgtgatggcaaggtgcttgttatgtctagaaagtaagaaaacaatatgaggagacattctgtctttcaaaaggtaatggtacatacgttcactggtctctaagtgtaaaagtagtaaattttgtgatgaataaaataattatctcctaattgtatgttagaataattttattagaataatttcatactgaaattattttctccaaataaaaattagatggaaaaatgtgaaaaaaattattcatgctctcatatatattttaaaaacactacttttgcttttttatttaccttttaagacattttcatgcttccaggtaaaaacagatattgtaccatgtacctaatccaaatatcatataaacattttatttatagttaataatctatgatgaaggtaattaaagtagattatggcctttttaagtattgcagtctaaaacttcaaaaactaaaatcattgtcaaaattaatatgattattaatcagaatatcagatatgattcactatttaaactatgataaattatgataatatatgaggaggcctcgctatagcaaaaatagttaaaatgctgacataacaccaaacttcattttttaaaaaatctgttgttccaaatgtgtataattttaaagtaatttctaaagcagtttattataatggtttgcctgcttaaaaggtataattaaacttcttttctcttctacattgacacacagaaatgtgtcaatgtaaagccaaaaccatcttctgtgtttatggccaatctattctcaaagttaaaagtaaaattgtttcagagtcacagttccctttatttcacataagcccaaactgatagacagtaacggtgtttagttttatactatatttgtgctatttaattctttctattttcacaattattaaattgtgtacactttcattacttttaaaaatgtagaaattcttcatgaacataactctgctgaatgtaaaagaaaattttttttcaaaaatgctgttaatgtatactactggtggttgattggttttattttatgtagcttgacaattcagtgacttaatatctattccatttgtattgtacataaaattttctagaaatacacttttttccaaagtgtaagtttgtgaatagattttagcatgatgaaactgtcataatggtgaatgttcaatctgtgtaagaaaacaaactaaatgtagttgtcacactaaaatttaattggatattgatgaaatcattggcctggcaaaataaaacatgttgaattcccc

SEQ ID NO: 14 - protein, 751 aa, Homo sapiens
MAFRTICVLVGVFICSICVKGSSQPQARVYLTFDELRETKTSEYFSLSHHPLDYRILLMDEDQDRIYVGSKDHILSLNINNISQEALSVFWPASTIKVEECKMAGKDPTHGCGNFVRVIQTFNRTHLYVCGSGAFSPVCTYLNRGRRSEDQVFMIDSKCESGKGRCSFNPNVNTVSVMINEELFSGMYIDFMGTDAAIFRSLTKRNAVRTDQHNSKWLSEPMFVDAHVIPDGTDPNDAKVYFFFKEKLTDNNRSTKQIHSMIARICPNDTGGLRSLVNKWTTFLKARLVCSVTDEDGPETHFDELEDVFLLETDNPRTTLVYGIFTTSSSVFKGSAVCVYHLSDIQTVFNGPFAHKEGPNHQLISYQGRIPYPRPGTCPGGAFTPNMRTTKEFPDDVVTFIRNHPLMYNSIYPIHKRPLIVRIGTDYKYTKIAVDRVNAADGRYHVLFLGTDRGTVQKVVVLPTNNSVSGELILEELEVFKNHAPITTMKISSKKQQLYVSSNEGVSQVSLHRCHIYGTACADCCLARDPYCAWDGHSCSRFYPTGKRRSRRQDVRHGNPLTQCRGFNLKAYRNAAEIVQYGVKNNTTFLECAPKSPQASIKWLLQKDKDRRKEVKLNERIIATSQGLLIRSVQGSDQGLYHCIATENSFKQTIAKINFKVLDSEMVAVVTDKWSPWTWASSVRALPFHPKDIMGAFSHSEMQMINQYCKDTRQQHQQGDESQKMRGDYGKLKALINSRKSRNRRNQLPES

SEQ ID NO: 15 - DNA, 6474 bp, Homo sapiens
  CDS (467)..(2794)
gtttggcaagtcagtgcaagaggctgacttctgagaggcttccaggagcccgaagagaggacctccacgggagaagggagtgcgtgtgctcggttttttttttttctctcttttttttttttttttctgaatgaacagctttgcccaagtgactgaaaaatacagcttcttcctgaatctaccggcgtagttgctgaagagcgctctagacaggacatggctctgaagactcactctttggaatgtcctcttgctcccggcttataaacaactgtcccgaggaaagaaaggttttacatagccaaatacagcctgacaaatggcacttcggaactgtgctttctgatgacaacgcgttcgatttctgacaaagcctctcgcacgctgcccctggagggaagtcctaagtaaaactcagaccctccttaaagtgaggagcgagggcttggacggtgaacacggcagcatggcatccgcggggcacattatcaccttgctcctgtggggttacttactggagctttggacaggaggtcatacagctgatactacccacccccggttacgcctgtcacataaagagctcttgaatctgaacagaacatcaatatttcatagcccttttggatttcttgatctccatacaatgctgctggatgaatatcaagagaggctcttcgtgggaggcagggaccttgtatattccctcagcttggagagaatcagtgacggctataaagagatacactggccgagtacagctctaaaaatggaagaatgcataatgaagggaaaagatgcgggtgaatgtgcaaattatgttcgggttttgcatcactataacaggacacaccttctgacctgtggtactggagcttttgatccagtttgtgccttcatcagagttggatatcatttggaggatcctctgtttcacctggaatcacccagatctgagagaggaaggggcagatgtccttttgaccccagctcctccttcatctccactttaattggtagtgaattgtttgctggactctacagtgactactggagcagagacgctgcgatcttccgcagcatggggcgactggcccatatccgcactgagcatgacgatgagcgtctgttgaaagaaccaaaatttgtaggttcatacatgattcctgacaatgaagacagagatgacaacaaagtatatttcttttttactgagaaggcactggaggcagaaaacaatgctcacgcaatttacaccagggtcgggcgactctgtgtgaatgatgtaggagggcagagaatactggtgaataagtggagcactttcctaaaagcgagactcgtttgctcagtaccaggaatgaatggaattgacacatattttgatgaattagaggacgtttttttgctacctaccagagatcataagaatccagtgatatttggactctttaacactaccagtaatatttttcgagggcatgctatatgtgtctatcacatgtctagcattcgggcagccttcaacggaccatatgcacataaggaaggacctgaataccactggtcagtctatgaaggaaaagtcccttatccaaggcctggttcttgtgccagcaaagtaaatggagggagatacggaaccaccaaggactatcctgatgatgccatccgatttgcaagaagtcatccactaatgtaccaggccataaaacctgcccataaaaaaccaatattggtaaaaacagatggaaaatataacctgaaacaaatagcagtagatcgagtggaagctgaggatggccaatatgacgtcttgtttattgggacagataatggaattgtgctgaaagtaatcacaatttacaaccaagaaatggaatcaatggaagaagtaattctagaagaacttcagatattcaaggatccagttcctattatttctatggagatttcttcaaaacggcaacagctgtatattggatctgcttctgctgtggctcaagtcagattccatcactgtgacatgtatggaagtgcttgtgctgactgctgcctggctcgagacccttactgtgcctgggatggcatatcctgctcccggtattacccaacaggcacacatgcaaaaaggcgtttccggagacaagatgttcgacatggaaatgcagctcagcagtgctttggacaacagtttgttggggatgctttggataagactgaagaacatctggcttatggcatagagaacaacagtactttgctggaatgtaccccacgatctttacaagcgaaagttatctggtttgtacagaaaggacgtgagacaagaaaagaggaggtgaagacagatgacagagtggttaagatggaccttggtttactcttcctaaggttacacaaatcagatgctgggacctatttttgccagacagtagagcatagctttgtccatacggtccgtaaaatcaccttggaggtagtggaagaggagaaagtcgaggatatgtttaacaaggacgatgaggaggacaggcatcacaggatgccttgtcctgctcagagtagcatctcgcagggagcaaaaccatggtacaaggaattcttgcagctgatcggttatagcaacttccagagagtggaagaatactgcgagaaagtatggtgcacagatagaaagaggaaaaagcttaaaatgtcaccctccaagtggaagtatgccaaccctcaggaaaagaagctccgttccaaacctgagcattaccgcctgcccaggcacacgctggactcctgatggggtgagactatctactgtcttttgaagaatttatatttggaaagtaaaaaagtaaaaaaataaatcatccaacttctttgcattacttaaaagagatttctgtaatacaggaatgactatgaaggtgttataataaattattctacatactcatttgactggataaactttacataaaattaactaattttttaaataaatgcattgcttaatggtttctcattatgtttatcaaaaaacaactgtagctgttattttcagtacttggctgcttttctgtgaaaattattattttacttttggaagacaagattattagaatattgaagaaaaattggagacttataatcatggtaaatataaaactaaatatgttttaatatttctgaatttttcttttccatcacaatgtaagatatgcagaatacaagatactttggcattctcatgtgaactttctgtactctttaaggattattttattagtgttgtttaagccatgagtgttaagtagcaggtgtgttgtgagtgctgtaacccatgaaaggaaaaatgtcattctgaggcttgtgcccttcgtaaaatattcattaaagtacattcacactatttttgctttataacacagtctttaattttcactcactgtggaaataaaaactaaggtaacttctcagaaagatatcaaatctcagaaagaatgtcaaatcagatgaagttatagttaggattctaactactgtaaaagatttttgcttccctcttgtggtaaaaaaaattatattctcacacatttcttttttctctacagacggatatctgtttaggaaagatttgaaagcagattatcagtaggtacatggatacatcaagttcatttgcagaaacaaataactgaaataaaaaacatgttaatccttgtatcatactttaatatgaaagtattgtttatagataatttatctcacaagtcaaaaatgaagattttgcagcactgaaaatctattaaagctccaaattttaagtttctaaataatcttcgctgaaatctaaaatatactataacaaccgtgttttatttgtgaaaaaaatattaaagtgatttgctctcaaatatcaaattttcttctctcttttatattaagagacagaaaattgtttcatgagttcacttaactactgagatattcagagcatttttacctctctcttaaatgttataaaaaacaattgtatttttaagaatgtttatttatcaaagtctttccttcttctattaaatatttagcaattacctttctaaaatatgaaattttgtaagatgttttcacctaaataaaaattgaaagcaagtggattacacaggagaaccattatgaacatttatttagatattaatcttaaacagtgtttatttcagttttcaaagttagcttataggttatacatttaagttaaagtgctcataatcacttgcaatttcattgtaaaatgaacaaatacataaatattttaagaaaaatttaagtttattcagataagtcaccatgcttcaaaagatctaagaaatgcaaatatactgaaaattgacatcctctgaaaattccacttgctatttacccaagaatccactggaggtcattactgccattaaataataactgaaaagactatgtagtgaaatgtatttttaaaaactatattcagtaaaagcctgctcaatttggagaaatagaaccacaaacacagatcacaggggccttacaaagtttatgtctgaacaaataagtcaattaagtacactttattgaaaattgccttccattaacacacaagaaagaaagcaggattttctcctgtatctgaattttaaaattaaaaaggcagataagacataaatagttatcattttaattgcaataacacagacaagtagttaatgatgataacaatggtgtaacttgtaaactaaatatttggtaactgaagcaataggcagaggaaaatagcttttctatgacacaagtcataagaagtccatatactgaagagcgtttgattaaaataaagtgactattaaccagaaaagaaacattttacataaaatgctaaaatttattataggaaaataaatcaaacccaaagaaagtttattcaatgctaatttgaaagaaaattgataagaaaactttgagggcccaagtccacaatttggtgagaccactaaattttacatataattatacacacacatatgtacatatatatgtatataatcttgcttcccgcctgtttatggcagtactgaagagaaatgggaaagaagagggagggagagagaaagacgaagggagagagaaagcagtttccaaggatatgtttcatgtcccaccattttctcagtttctccctctctctcccaacacacacacacacacacccctcacatactataaaataaatcttcactgccctatcaaaatacaaataaatcaatctatgctgttctgtccttcttgagaatctaaaacataccacaaaaatacatccccagtcttttgttctgtctgaggttagaattaattcaaattcagaatctgttgtgagaaatgcccaggctttaaaaattaaaaatggatggatcttctctgaactcagggagggcacatacttagatacctacaagacttggaggaattaagagttcacccttcatctcaccaaattttccccatttttctctttcttgtagaaggagagaaaccatgctctctagcaacattgagcaaaaatcataaccactcatctaatttctaagaggcacctccatcgagggccggtctcctgcttctttagacctcttctatctttgttacaggagaggacctgtggatagacttagttttgacataaaacaatgcccattcacctcctccttcagcacaacgtcacccattgggcaagagatccagatttgttaacaaaaaagattttacttcgtgattccacgtctataattctatattgctaattttttcttttgtgtgaattactgaatatttcagagcaaagctatcaacttggagaaacagggattaaaaataaggataaacactaataagagctctagaaaaaagggaacagaaagtctgcctgtttagtaagtggcaattccatacatattttagagttttttctatctaaaattagttaaatacttagaatgtttgtaatgagtgttcgatatttgctataggttttagggttttgtaaatcttcatagtaattataaacatttgtaaaatttgtaaaatactataagtcattttgagtgttggtgttaagcatgaaacaaacagcagctgttgtccttaaaaatgaattgacctggccgggcgcggtggctcacgcctgtaatcccagcactttgggaggccgaggcgggtggatcatgaggtcaggagatggagaccatcctggctaacaaggtgaaaccccgtctctactaaaaatacaaaaaattagccgggcgcggtggcgggcgcctgtagtcccagctacttgggaggctgaggcaggagaatggcgtgaacccgggaagcggagcttgcagtgagccgagattgcgccactgcagtccgcagtccggcctgggcgacagagcgagactccgtctc

SEQ ID NO: 16 - protein, 775 aa, Homo sapiens
MASAGHIITLLLWGYLLELWTGGHTADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 17 - DNA, 2719 bp, Homo sapiens
  CDS (79)..(2436)
cggggcccaggccccgccgctgcggaagaggtttctagagagtggagcctgcttcctgggccctaggcccctcccacaatgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcaggcgacgggcgctatgaggtgcttttcctgggcacagaccgcgggacagtgcagaaggtcattgtgctgcccaaggatgaccaggagatggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacacatgaggccagctgcctgtgcctgccatgggccaggctaggccttggtcccttttaatataaaagatatatatatatatatatatatatattaaaatatcggggtggggggtgattggaagggagggaggtggccttcccaatgcgcgttattcggggttattgaagaataatattgcaagtgacagccagaagtagactttctgtcctcacaccgaagaacccgagtgagcaggagggagggagagacgcgaagagaccttttttcctttttggagaccttgtccgc

SEQ ID NO: 18 - protein, 785 aa, Homo sapiens
MLVAGLLLWASLLTGAWPSFPTQDHLPATPRVRLSFKELKATGTAHFFNFLLNTTDYRILLKDEDHDRMYVGSKDYVLSLDLHDINREPLIIHWAASPQRIEECVLSGKDVNGECGNFVRLIQPWNRTHLYVCGTGAYNPMCTYVNRGRRAQATPWTQTQAVRGRGSRATDGALRPMPTAPRQDYIFYLEPERLESGKGKCPYDPKLDTASALINEELYAGVYIDFMGTDAAIFRTLGKQTAMRTDQYNSRWLNDPSFIHAELIPDSAERNDDKLYFFFRERSAEAPQSPAVYARIGRICLNDDGGHCCLVNKWSTFLKARLVCSVPGEDGIETHFDELQDVFVQQTQDVRNPVIYAVFTSSGSVFRGSAVCVYSMADIRMVFNGPFAHKEGPNYQWMPFSGKMPYPRPGTCPGGTFTPSMKSTKDYPDEVINFMRSHPLMYQAVYPLQRRPLVVRTGAPYRLTTIAVDQVDAGDGRYEVLFLGTDRGTVQKVIVLPKDDQEMEELMLEEVEVFKDPAPVKTMTISSKRQQLYVASAVGVTHLSLHRCQAYGAACADCCLARDPYCAWDGQACSRYTASSKRRSRRQDVRHGNPIRQCRGFNSNANKNAVESVQYGVAGSAAFLECQPRSPQATVKWLFQRDPGDRRREIRAEDRFLRTEQGLLLRALQLSDRGLYSCTATENNFKHVVTRVQLHVLGRDAVHAALFPPLSMSAPPPPGAGPPTPPYQELAQLLAQPEVGLIHQYCQGYWRHVPPSPREAPGAPRSPEPQDQKKPRNRRHHPPDT

SEQ ID NO: 19 - DNA, 649 bp, Homo sapiens
  CDS (17)..(592)
  misc_feature (17)..(94): Signal peptide
tcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagcgtttcgggaaccagatctctcaccagg

SEQ ID NO: 20 - protein, 191 aa, Homo sapiens
  misc_feature (17)..(94): Signal peptide
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 21 - DNA, 755 bp, Homo sapiens
  CDS (5)..(628)
caccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaa

SEQ ID NO: 22 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 23 - DNA, 1997 bp, Homo sapiens
  CDS (352)..(1611)
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 24 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 25 - DNA, 2029 bp, Homo sapiens
  CDS (411)..(1475)
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 26 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 27 - DNA, 1645 bp, Homo sapiens
  CDS (322)..(771)
gggattcgggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggattctgctggggccgccacggcctggtggtgggaaggccggcagcgggcggaggggattcagccacttccccctcttcttctgaagatcagaacattcagctctggagaacagtggttgcctgggggcttttgccactccttgtcccccgtgatctcccctcacactttgccatttgcttgtactgggacattgttctttccggccgaggtgccaccaccctgcccccactaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgc

SEQ ID NO: 28 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 29 - DNA, 4230 bp, Homo sapiens
  CDS (1)..(4065)
agcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacgacggtgattgccatgttcttctggctacttcttgtcatcatcctagggaccgttaagcgggccaatggaggggaactgaagacaggctacttgtccatcgtcatggatccagatgaactcccattggatgaacattgtgaacgactgccttatgatgccagcaaatgggaattccccagagaccggctgaacctaggtaagcctcttggccgtggtgcctttggccaagagattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgacacggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagctcttctaggcttgtgacc

SEQ ID NO: 30 - protein, 1354 aa, Homo sapiens
SKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTTVIAMFFWLLLVIILGTVKRANGGELKTGYLSIVMDPDELPLDEHCERLPYDASKWEFPRDRLNLGKPLGRGAFGQEIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDTGTTLSSPPV

SEQ ID NO: 31 - DNA, 4195 bp, Homo sapiens
  CDS (20)..(3913)
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaaggggttaggcctccggatgactgcgggcaggcctggataatatccagcctcccacaagaagctggtggagcagagtgttccctgactcct

SEQ ID NO: 32 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 33 - protein, 14 aa, Homo sapiens
GKPIPNPLLGLDST